SEQUENCE LISTING (10 sequences)

SEQ ID NO: 1 - protein, 2351 aa, Artificial Sequence
  Synthetic Construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 3 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 4 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 5 - DNA, 17 bp, Artificial Sequence
  Synthetic Construct
ggcgggcgactcagatc

SEQ ID NO: 6 - DNA, 21 bp, Artificial Sequence
  Synthetic Construct
gggaggctgctggtgaatatt

SEQ ID NO: 7 - DNA, 27 bp, Artificial Sequence
  Synthetic Construct
agcccctgtttgctcctccgataactg

SEQ ID NO: 8 - DNA, 21 bp, Artificial Sequence
  Synthetic Construct
aaaacgagcagtgacgtgagc

SEQ ID NO: 9 - DNA, 20 bp, Artificial Sequence
  Synthetic Construct
ttcagtcatgctgctagcgc

SEQ ID NO: 10 - DNA, 25 bp, Artificial Sequence
  Synthetic Construct
tgcacggaagcgtctcgtctcagtc

(19) United States
(12) Patent Application Publication
(10) Pub. No.: US 2020/0338216 A1
(43) Pub. Date: Oct. 29, 2020

(54) IMMUNO-EVASIVE VECTORS AND USE FOR GENE THERAPY

(71) Applicant: CHAMELEON BIOSCIENCES, INC., Berkeley, CA (US)

(72) Inventor: Genine WINSLOW, San Anselmo, CA (US)

(21) Appl. No.: 16/961,500

(22) PCT Filed: Jan. 11, 2019
(86) PCT No.: PCT/US19/13361

§ 371 Date: Jul. 10, 2020

Related U.S. Application Data
(60) Provisional application No. 62/616,167, filed on Jan. 11, 2018.
(60) Provisional application No. 62/768,779, filed on Nov. 16, 2018.

(51) Int. Cl. A61K 48/00; C12N 15/86

(52) U.S. Cl. CPC A61K 48/0083; C12N 15/86; A61K 9/127; C12N 2740/16043; C12N 2740/15043; C12N 2750/14143

(57) ABSTRACT

Provided is an enveloped viral vector comprising a viral particle surrounded by an envelope, wherein the viral particle comprises a heterologous transgene, and the envelope comprises a lipid bilayer and one or more immunosuppressive molecules, and methods for preparing and using same.

### CROSS-REFERENCE TO RELATED APPLICATIONS

This application claims the priority benefit of U.S. Provisional Application Nos. 62/616,167, filed Jan. 11, 2018 and U.S. Provisional Application Nos. 62/768,779, filed Nov. 16, 2018, the entire disclosures of which are hereby incorporated by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 774392000140SeqList.txt, date recorded: Jan. 11, 2019, size: 29 KB).

### FIELD OF THE INVENTION

The present disclosure relates generally to improved vectors for gene therapy with reduced immunogenicity.

### BACKGROUND

AAV Gene Therapy clinical trials have shown that AAV can be safely used to reverse disease phenotypes for several monogenic diseases including Spinal Muscular Atrophy (SMA) (Meliani et al. (2017) Blood Advances, 1(23): 2019-31), Hemophilia B (Nathwani et al. (2011) N Engl J Med, 365: 2357-65), and inherited retinal diseases caused by mutations in the RPE65 gene (Simonelli et al. (2010) Molecular Therapy, 18(3): 643-650). In addition to promising human clinical trial data there are also further examples of promising pre-clinical data using AAV gene therapy; for example, Myotubularin Myopathy (Childers et al. (2014) Sci Transl Med, 6: 220ra10). Despite positive clinical and pre-clinical data, the immune response generated to recombinant AAV and or the newly expressed therapeutic protein remains a barrier to more widespread use of AAV gene therapy for treating monogenic disorders (Mingozzi et al. (2013) Blood, 122(1): 23-36; Chermule et al. (1999) Gene Therapy; 6, 1574-1583; Masat et al. (2013) Discov Med, 15(85): 379-389).

While it has become apparent that AAV based gene therapy promises to be curative, there are questions surrounding the longevity of a single treatment. There is evidence of AAV mediated delivery of therapeutic protein function for up to three years (Nathwani et al. (2014) N Engl J Med, 371: 1994-2004), but lifetime transgene expression has yet to be proven, and in some cases is unlikely. AAV based vectors persist as episomal elements, double stranded DNA loop structures that do not integrate into cell chromosomes. For this reason, AAV genomes do not replicate and divide as a cell divides and can be diluted out by cell division. To insure prolonged transgene expression, AAV gene therapy investigators have targeted cell types that divide slowly or do not divide at all; for example muscle, liver, or neuronal cells. It is therefore unknown whether AAV delivered therapeutic genes will be expressed for the lifetime of the patient. This is especially true in life threatening diseases that affect young children such as Spinal Muscular Atrophy, because the muscle cells of young children will undergo more cell division as the child grows than would adult muscle cells. While clinical data suggests that AAV delivery of a therapeutic gene can improve defined SMA disease endpoints, it is unlikely that expression levels will be maintained for the life of the child. Indeed, even adults receiving AAV gene therapy for slowly or non-dividing cells will likely experience a reduction in therapeutic protein levels over the lifetime of the patient, due to the diluting out of the AAV genomes in transduced cells. It would therefore be advantageous to be able to deliver additional doses of AAV gene therapy products.

The host immune response to AAV gene therapy remains a hurdle that must be overcome before AAV gene therapy may be used more widely. Because AAV is a naturally occurring virus, portions of patient populations have pre-existing antibodies to different AAV serotypes. For example, pre-existing antibodies to AAV2, the most common serotype, can be found in up to 60% of the population (Chiermule et al (1999) Gene Therapy; 6, 1574-1583). Other AAV serotypes are less common, but can't be utilized to target all tissue types; for example AAV5 preferentially infects the liver and AAV8 preferentially targets muscle cells (Asokan et al. (2012) Molecular Therapy, 20 (4) 699-708). A next generation AAV Vector that can be selectively targeted to specific tissues while evading the pre-existing antibodies to AAV would increase the potential patient population and enable the use of a single manufacturing platform to address vectors for multiple disease targets.

Host immune responses to AAV gene therapy prevent administration of second doses of product due to capsid specific adaptive immune responses. Additionally, a T cell response to novel expression of a therapeutic protein may reduce efficacy of AAV gene therapy products (Mingozzi et al. (2013) Blood, 122(1): 23-36).

Efforts have been made to reduce the effect of host immune response on AAV therapy. For instance, enveloped-AAV (also known as “exo-AAV”) have been shown to be more effective than non-enveloped AAV it is believed due to shielding the vector to some extent from the ability of anti-AAV antibodies to clear vector in vivo and in vitro (Gyorgy et al. (2014) Biomaterials, 35(26): 7598-7609; Hudry et al. (2016) Gene Ther, April, 23(4): 380-92; US 2013/020559). Also, there is some evidence that co-administration of an AAV encoding PD-L1 or PD-L2 with CTLA-4-Ig prolongs transgene expression and results in fewer transgene responsive T Cells (Adriouch et al. (2011) Front Microbiol, 2:199). The present invention uses enveloped AAV technology combined with checkpoint immune modulating molecules to create Effector Vectors to reduce the immune response and restrictions in dosing, and to facilitate repeat dosing of a therapeutic gene.

Still, there remains a need for new viral vectors and methods that improve transgene delivery and expression while minimizing the effect of the host immune response.

All references cited herein, including patent applications and publications, are incorporated by reference in their entirety.

### BRIEF SUMMARY OF THE INVENTION

Provided herein is an enveloped viral vector comprising a vector particle surrounded by an envelope, wherein the vector particle comprises a transgene and the envelope comprises one or more immunosuppressive molecules. Also provided is a pharmaceutical composition comprising the enveloped viral vector and one or more pharmaceutically acceptable carriers or excipients.

Also provided is a method of delivering a transgene to a cell or subject comprising administering the enveloped viral vector to the cell or subject, as well as a method of treating a disease or disorder in a subject by administering the enveloped viral vector to the subject.

Further provided is a method of producing the enveloped viral vector, the method comprising (a) culturing a viral producer cells (i.e., in vitro) under conditions to generate enveloped viral particles, wherein the viral producer cells comprise nucleic acids encoding one or more one or more membrane bound immunosuppressive molecules, and (b) collecting the enveloped viral vectors.

In some aspects, the invention provides a composition comprising an enveloped viral vector, wherein the enveloped viral vector comprises a vector particle surrounded by envelop, wherein the envelope comprises one or more molecules that provide immune effector functions. In some embodiments, the immune effector functions reduce immunogenicity of the enveloped vector compared to a vector without immune effector molecules. In some embodiments, the immune effector functions stimulate immune inhibitors. In other embodiments, the immune effector functions inhibit immune stimulating molecules. In some embodiments, the envelope comprises molecules that stimulate immune inhibitors and molecules that inhibit immune stimulating molecules. In some embodiments, the one or more molecules providing immune effector functions includes, but is not limited to, one or more of CTLA4, B7-1, B7-2, PD-1, PD-L1, PD-L2, CD28, or VISTA. In some embodiments, the envelope comprises CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-L1 and VISTA. In some embodiments, the one or more molecules that provides immune effector functions comprises a transmembrane domain. In some embodiments, the envelope further comprises targeting molecules that target the vector to one or more cell types. In some embodiments, the targeting molecules confer tissue specificity to the enveloped vector. In some embodiments, the targeting molecule is an antibody. In some embodiments, the antibody is antibody 8D7. In some embodiments, the one or more targeting molecules comprise a transmembrane domain.

In some embodiments of the above aspects and embodiments, the viral vector comprises a viral particle. In some embodiments, the viral particle comprises a viral capsid and a viral genome. In some embodiments, the viral genome comprises one or more heterologous transgenes. In some embodiments, the heterologous transgene encodes a polypeptide. In some embodiments, the heterologous transgene encodes a therapeutic polypeptide or a reporter polypeptide. In some embodiments, the therapeutic polypeptide is Factor VIII, Factor IX, myotubularin, SMN, RPE65, NADH-ubiquinone oxidoreductase chain 4, CHM, huntingtin, alpha-galactosidase A, acid beta-glucosidase, alpha-glucosidase, ornithine transcarbomylase, argininosuccinate synthetase, β-globin, γ-globin, phenylalanine hydroxylase, or ALD. In some embodiments, the heterologous transgene encodes a therapeutic nucleic acid. In some embodiments, the therapeutic nucleic acid is a siRNA, miRNA, shRNA, antisense RNA, RNAzyme, or DNAzyme. In some embodiments, the heterologous transgene encodes one or more gene editing gene products. In some embodiments, the one or more gene editing gene products is a CAS nuclease and/or one or more guide sequences and/or one or more donor sequences.

In some embodiments of the above aspects and embodiments, the viral vector is an adeno-associated viral (AAV) vector or a lentiviral vector. In some embodiments, the viral vector is an adeno-associated viral vector. In some embodiments, the AAV vector comprises a capsid from human AAV serotype AAV1, AAV2, AAV3, AAV4, AAV5, AAV6, AAV7, AAV8, AAV9, AAV10, AAV11 or AAV12. In some embodiments, the AAV vector comprises an AAV viral genome comprising inverted terminal repeat (ITR) sequences from human AAV serotype AAV1, AAV2, AAV3, AAV4, AAV5, AAV6, AAV7, AAV8, AAV9, r AAV10. In some embodiments, the AAV capsid and the AAV ITR are from the same serotype or from different serotypes.

In some embodiments of the above aspects and embodiments, the viral vector is a lentiviral vector. In some embodiments, the lentiviral vector is derived from human immunodeficiency virus, a simian immunodeficiency virus or a feline immunodeficiency virus. In some embodiments, the lentiviral vector is non-replicating. In some embodiments, the lentiviral vector is non-integrating.

In some embodiments, the invention provides a pharmaceutical composition comprising any of the composition described above and one or more pharmaceutically acceptable excipients.

In some aspects, the invention provides a method of delivering a transgene to an individual comprising administering a composition comprising an enveloped viral vector to the individual, wherein the enveloped viral vector comprises a vector particle surrounded by envelop, wherein the envelope comprises one or more molecules that provide immune effector functions and wherein the viral particle comprises a viral genome comprising the transgene. In some aspects, the invention provides a method of treating an individual with a disease or disorder comprising administering a composition comprising an enveloped viral vector to the individual in need thereof, wherein the enveloped viral vector comprises a vector particle surrounded by envelop, wherein the envelope comprises one or more molecules that provide immune effector functions and wherein the viral particle comprises a viral genome comprising a therapeutic transgene. In some embodiments, the immune effector functions reduce immunogenicity of the enveloped vector compared to a vector without immune effector molecules. In some embodiments, the immune effector functions stimulate immune inhibitors. In other embodiments, the immune effector functions inhibit immune stimulating molecules. In some embodiments, the envelope comprises molecules that stimulate immune inhibitors and molecules that inhibit immune stimulating molecules. In some embodiments, the one or more molecules providing immune effector functions includes one or more of CTLA4, B7-1, B7-2, PD-1, PD-L1, PD-L2, CD28, or VISTA. In some embodiments, the envelope comprises CTLA4 and PD-L1 or CTLA and PD-L2. In some embodiments, the one or more molecules that provides immune effector functions comprises a transmembrane domain. In some embodiments, the envelope further comprises targeting molecules that target the vector to one or more cell types. In some embodiments, the targeting molecules confer tissue specificity to the enveloped vector. In some embodiments, the targeting molecule is an antibody. In some embodiments, the antibody is antibody 8D7. In some embodiments, the one or more targeting molecules comprise a transmembrane domain.

In some embodiments of the above methods, the viral vector comprises a viral particle. In some embodiments, the viral particle comprises a viral capsid and a viral genome. In some embodiments, the viral genome comprises one or more heterologous transgenes. In some embodiments, the heterologous transgene encodes a polypeptide. In some embodiments, the heterologous transgene encodes a therapeutic polypeptide or a reporter polypeptide. In some embodiments, the therapeutic polypeptide is Factor VIII, Factor IX, myotubularin, SMN, RPE65, NADH-ubiquinone oxidoreductase chain 4, CHM, huntingtin, alpha-galactosidase A, acid beta-glucosidase, alpha-glucosidase, ornithine transcarbomylase, argininosuccinate synthetase, β-globin, γ-globin, phenylalanine hydroxylase, or ALD. In some embodiments, the heterologous transgene encodes a therapeutic nucleic acid. In some embodiments, the therapeutic nucleic acid is a siRNA, miRNA, shRNA, antisense RNA, RNAzyme, or DNAzyme. In some embodiments, the heterologous transgene encodes one or more gene editing gene products. In some embodiments, the one or more gene editing gene products is a CAS nuclease and/or one or more guide sequences and/or one or more donor sequences.

In some embodiments of the above methods, the viral vector is an adeno-associated viral (AAV) vector or a lentiviral vector. In some embodiments, the viral vector is an adeno-associated viral vector. In some embodiments, the AAV vector comprises a capsid from human AAV serotype AAV1, AAV2, AAV3, AAV4, AAV5, AAV6, AAV7, AAV8, AAV9, AAV10, AAV11 or AAV12. In some embodiments, the AAV vector comprises an AAV viral genome comprising inverted terminal repeat (ITR) sequences from human AAV serotype AAV1, AAV2, AAV3, AAV4, AAV5, AAV6, AAV7, AAV8, AAV9, r AAV10. In some embodiments, the AAV capsid and the AAV ITR are from the same serotype or from different serotypes.

In some embodiments of the above methods, the viral vector is a lentiviral vector. In some embodiments, the lentiviral vector is derived from human immunodeficiency virus, a simian immunodeficiency virus or a feline immunodeficiency virus. In some embodiments, the lentiviral vector is non-replicating. In some embodiments, the lentiviral vector is non-integrating.

In some embodiments of the above methods, the composition is a pharmaceutical composition comprising enveloped viral vector and one or more pharmaceutically acceptable excipients.

In some embodiments of the above methods, the individual is a human. In some embodiments, the disease or disorder is monogenic disease. In some embodiments, the disease or disorder is myotobularin myopathy, spinal muscular atrophy, Leber's congenital amaurosis, hemophilia A, hemophilia B, choroideremia, Huntington's disease, Batten disease, Leber hereditary optic neuropathy, ornithine transcarbamylase (OTC) deficiency, Pompe disease, Fabry disease, citrullinemia type 1, phenylketonuria (PKU), adrenoleukodystrophy, sickle cell disease, or beta thalessemia.

In some aspects, the invention provides a method of producing an enveloped viral vector with reduced immunogenicity, the method comprising a) culturing a viral producer cells under conditions to generate enveloped viral particles, wherein the viral producer cells comprise nucleic acid encoding one or more one or more membrane bound immune effector functions that reduce immunogenicity of the enveloped vector, and b) collecting the enveloped viral vectors. In some embodiments, the immune effector functions reduce immunogenicity of the enveloped vector. In some embodiments, the immune effector functions stimulate immune inhibitors. In some embodiments, the immune effector functions inhibit immune stimulating molecules. In some embodiments, the viral producer cells comprise nucleic acid encoding molecules that stimulate immune inhibitors and molecules that inhibit immune stimulating molecules. In some embodiments, the one or more molecules providing immune effector functions includes one or more of CTLA4, B7-1, B7-2, PD-1, PD-L1, PD-L2, CD28, or VISTA. In some embodiments, the viral producer cells comprise nucleic acid encoding CTLA4 and PD-L1 or CTLA and PD-L2. In some embodiments, the one or more molecules that provide immune effector functions comprises a transmembrane domain. In some embodiments, nucleic acid encoding the one or more molecules providing immune effector functions is transiently introduced to the viral producer cells. In some embodiments, nucleic acid encoding the one or more molecules providing immune effector functions is stably maintained in the viral producer cells. In some embodiments, nucleic acid encoding the one or more molecules providing immune effector functions is integrated into the genome of the viral producer cell.

In some embodiments of the above methods, the viral producer cells comprise nucleic acid encoding one or more targeting molecules that target the vector to one or more cell types. In some embodiments, the targeting molecules confer tissue specificity to the enveloped vector. In some embodiments, the targeting molecule is an antibody. In some embodiments, the antibody is antibody 8D7. In some embodiments, the one or more targeting molecules comprise a transmembrane domain. In some embodiments, nucleic acid encoding the one or more targeting molecules is transiently introduced to the viral producer cells. In some embodiments, nucleic acid encoding the one or more targeting molecules is stably maintained in the viral producer cells. In some embodiments, nucleic acid encoding the one or more molecules targeting molecules is integrated into the genome of the viral producer cell.

In some embodiments of the above methods, the enveloped viral vector is an enveloped AAV vector. In some embodiments, the viral producer cells comprise a) nucleic acid encoding AAV rep and cap genes, b) nucleic acid encoding an AAV viral genome comprising a transgene and at least one ITR, and c) AAV helper functions. In some embodiments, the nucleic acid encoding AAV rep and cap genes and/or the AAV viral genome are transiently introduced in the producer cell line. In some embodiments, the nucleic acid encoding AAV rep and cap genes and/or the AAV viral genome are stably maintained in the producer cell line. In some embodiments, the nucleic acid encoding AAV rep and cap genes and/or the AAV viral genome are stably integrated into the genome of the producer cell line. In some embodiments, the rAAV genome comprises two AAV ITRs. In some embodiments, one or more AAV helper functions are provided by one or more of a plasmid, an adenovirus, a nucleic acid stably integrated into the cell genome or a herpes simplex virus (HSV). In some embodiments, AAV helper functions comprise one or more of adenovirus E1A function, adenovirus E1B function, adenovirus E2A function, adenovirus E4 function and adenovirus VA function. In some embodiments, AAV helper functions comprise one or more of HSV UL5 function, HSV UL8 function, HSV UL52 function, and HSV UL29 function.

In some embodiments of the above methods, the enveloped viral vector is a lentiviral vector. In some embodiments, the lentiviral vector is a human immunodeficiency virus, a simian immunodeficiency virus or a feline immunodeficiency virus. In some embodiments, the viral producer cells comprise a) nucleic acid encoding lentiviral gag gene, b) nucleic acid encoding lentiviral pol gene, c) nucleic acid encoding a lentiviral transfer vector comprising a transgene, a 5′ long terminal repeat (LTR) and a 3′ LTR, wherein all or part of a U3 region of the 3′ LTR is replaced by a heterologous regulatory element, a primer binding site, all or part of the GAG gene, a central polypurine tract, synthetic stop codons in the GAG sequence, rev responsive element, and an env splice acceptor.

In some embodiments of the above methods, the enveloped vector is further purified.

In some aspects, the invention provides a kit comprising the any of the compositions described herein. In some embodiments, the kit of further comprising instructions for use.

In some aspects, the invention provides a composition for use in delivering a nucleic acid to an individual in need thereof according to any of the methods described herein. In some embodiments, the invention provides a composition for use in treating a disease or disorder to an individual in need thereof according to any of the methods described herein. In some embodiments, the invention provides the use of the composition as described herein in the manufacture of a medicament for delivering a nucleic acid to an individual in need thereof. In some embodiments, the invention provides the use of the composition as described herein in the manufacture of a medicament for treating an individual with a disease or disorder. In some embodiments, the disease or disorder is myotobularin myopathy, spinal muscular atrophy, Leber's congenital amaurosis, hemophilia A, hemophilia B, choroideremia, Huntington's disease, Batten disease, Leber hereditary optic neuropathy, ornithine transcarbamylase (OTC) deficiency, Pompe disease, Fabry disease, citrullinemia type 1, phenylketonuria (PKU), adrenoleukodystrophy, sickle cell disease, or beta thalessemia.

In some aspects, the invention provides an article of manufacture comprising the composition as described herein.

Additional compositions and methods are provided as described in the following detailed description of the invention.

### BRIEF DESCRIPTION OF THE DRAWINGS

FIG. 1 shows an exemplary schematic of an effector vector. In this particular example, an AAV vector is enveloped in a cell membrane engineered to present immune effector functions as well as cell targeting functions on the surface of the enveloped viral particle.

FIG. 2 shows an exemplary effector molecule.

FIG. 3 shows the presence of mouse PDL1 (left panel) and mouse CTLA4 on the envelopes of EVADER vectors. FACS histograms show enveloped AAV and EVADER Vectors stained with anti-mouse PDL1or anti-mouse CTLA-4 antibodies. The enveloped AAV histograms are superimposed on the Effector Vector histograms to show higher levels of PDL-1 or CTLA-4 staining of purified vectors. Effector Vectors have higher levels of both PDL-1 and CTLA-4 than enveloped AAV vectors. EVADER is Effector Vectors.

FIG. 4 shows graphs showing human FIX levels in mice at 3 and 6 weeks post initial injection. For 3 week female mice: *p=0.036; **p+0.002; ****p<0.0001. For 3 week male mice: ****p<0.0001. For 6 week female mice: std vs. evader p=0.0002; exo vs. evader p=0.0006. Evader is mEV-AAV-hFIX. Exo is enveloped AAV.

FIG. 5 shows graphs of titers of anti-AAV8 IgG antibodies in serum from mice at weeks 3 and 6.

FIG. 6 shows graphs of titers of neutralizing antibodies to AAV8 at weeks 3 and 6.

FIG. 7 shows graphs depicting vector genome copy numbers (VGCN) from livers of male of female mice at weeks 3 and 6.

FIG. 8 shows graphs depicting vector genome copy numbers (VGCN) from livers of combined male and female mice at weeks 3 and 6.

FIG. 9 shows graphs depicting vector genome copy numbers (VGCN) from livers of combined male and female mice at week 6 including statistical analysis.

### DETAILED DESCRIPTION

Provided herein is an enveloped viral vector comprising a viral particle surrounded partially or completely by an envelope, wherein the envelope comprises a lipid bilayer and one or more immune-suppressing molecules, such as checkpoint immune down-regulators. In some embodiments, enveloped viruses (e.g., AAV or lentivirus) are produced by “budding” off from the viral producer cell membranes. Immune modulating molecules imbedded in producer cell membranes are, therefore, transferred to the enveloped virus because the envelope comprises a portion of the producer cell membrane. As described in detail in the following sections, the enveloped viral vector is useful for delivering a nucleic acid (transgene) to a cell or subject, and is believed to be resistant to host-generated immune response. The enveloped viral vector and methods for its use and production are described in detail in the following sections.

### I. GENERAL TECHNIQUES

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### II. DEFINITIONS

For purposes of interpreting this specification, the following definitions will apply unless otherwise stated. Whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

It is understood that aspects and embodiments of the disclosure described herein include “comprising,” “consisting,” and “consisting essentially of” such aspects and embodiments.

For all compositions described herein, and all methods using a composition described herein, the compositions can either comprise the listed components or steps, or can “consist essentially of” or “consist of” the listed components or steps. When a composition is described as “consisting essentially of” the listed components, the composition contains the components listed, and may contain other components which do not substantially affect the methods disclosed, but do not contain any other components which substantially affect the methods disclosed other than those components expressly listed; or, if the composition does contain extra components other than those listed which substantially affect the methods disclosed, the composition does not contain a sufficient concentration or amount of the extra components to substantially affect the methods disclosed. When a method is described as “consisting essentially of” the listed steps, the method contains the steps listed, and may contain other steps that do not substantially affect the methods disclosed, but the method does not contain any other steps which substantially affect the methods disclosed other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the properties of composition or the methods disclosed.

The term “about” as used herein refers to the usual error range for the respective value readily known to the skilled person in this technical field. Reference to “about” a value or parameter herein includes (and describes) embodiments that are directed to that value or parameter per se.

The term “polynucleotide” or “nucleic acid” as used herein refers to a polymeric form of nucleotides of any length, ribonucleotides, deoxyribonucleotides or combination therein. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate- phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to any particular minimum or maximum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “viral vector” refers to a polynucleotide vector comprising one or more heterologous sequences (i.e., nucleic acid sequence not of viral origin) that are flanked by at least one or two repeat sequences (e.g., inverted terminal repeat sequences (ITRs) for AAV or long terminal repeats (LTRs) for lentivirus). The heterologous nucleic acid and be referred to as a “payload” to be delivered as a “cassette” and is often flanked by the at least one or two repeat sequences (e.g., inverted terminal repeat sequences (ITRs) for AAV or long terminal repeats (LTRs) for lentivirus). Such viral vectors can be replicated and packaged into infectious viral particles when present in a host cell provided that the host cell provides the essential functions. When a viral vector is incorporated into a larger polynucleotide (e.g., in a chromosome or in another vector such as a plasmid used for cloning or transfection), then the viral vector may be referred to as a “pro-vector” which can be “rescued” by replication and encapsidation in the presence of viral replication and packaging functions. A viral vector can be packaged into a virus capsid to generate a “viral particle”. In some respects, a viral particle refers to a virus capsid together with the viral genome and heterologous nucleic acid payload.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector. A heterologous nucleic acid may refer to a nucleic acid derived from a genotypically distinct entity from that of the rest of the entity to which it is compared or into which it is introduced or incorporated. Heterologous also can be used to refer to other biological components (e.g., proteins) that are non-native to the species into which they are introduced. For instance, a protein expressed in a cell from a heterologous nucleic acid would be a heterologous protein with respect to the cell. A nucleic acid introduced into a cell or organism by genetic engineering techniques may be considered “exogenous” to the cell or organism regardless of whether it is heterologous or homologous to the cell or organism. Thus, for instance, a vector could be used to introduce an additional copy of human gene into a human cell. The gene introduced to the cell would be exogenous to the cell even though it might contain a homologous (native) nucleic acid sequence.

An “isolated” molecule (e.g., nucleic acid or protein) or cell means it has been identified and separated and/or recovered from a component of its natural environment.

“Engineered” or “genetically engineered” and like terms are used to refer to biological materials that are artificially genetically modified (e.g., using laboratory techniques) or result from such genetic modifications.

As used herein, “treatment” is an approach for obtaining beneficial or desired clinical results. For purposes of this invention, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

An “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

For any of the structural and functional characteristics described herein, methods of determining these characteristics are known in the art.

### III. VECTORS

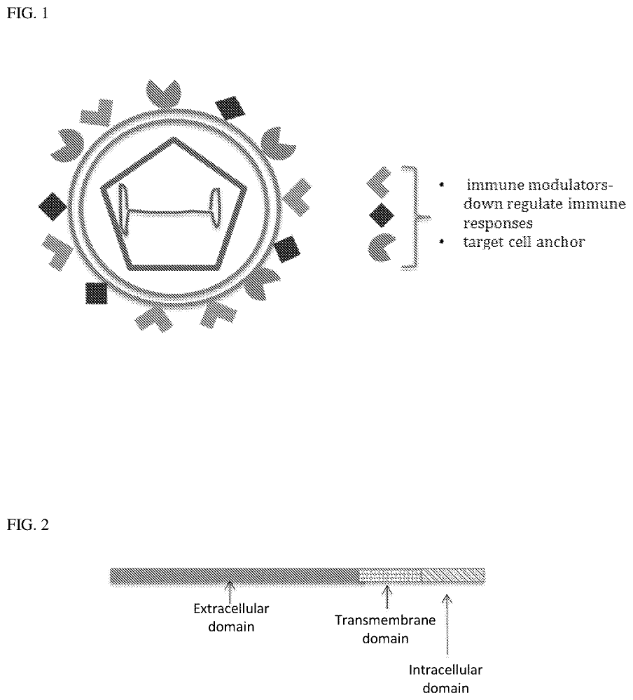

Provided herein is an enveloped viral vector comprising a viral particle surrounded partially or completely by an envelope, wherein the envelope comprises a lipid bilayer and one or more immune-suppressing molecules. A schematic depiction of an effector vector is shown in FIG. 1. In some embodiments, the enveloped viral vectors provided herein can deliver a nucleic acid transgene payload more effectively and/or more efficiently than the same enveloped vector without an envelope or with an envelope that is not engineered to include immunosuppressive molecules in the envelope.

In some embodiments, the enveloped viral particles are engineered for reduced immunity to the viral particle compared to the native viral particle. In some embodiments, the enveloped viral particles are engineered for reduced immunity to the viral transgene product compared to a vector comprising a native viral particle encoding a transgene product. In some embodiments, the enveloped viral particle is not enveloped in its typical native state; e.g. adeno-associated virus (AAV) particles and adenoviral particles. In other embodiments, the native viral particle is enveloped; for example, retroviruses and herpes viruses, where the envelope is engineered to modulate immunity to the viral particle and/or viral transgene product.

For instance, in some embodiments, the enveloped viral vector (e.g., enveloped AAV) comprising immunosuppressive molecules in the envelope, as provided herein, provides transgene expression levels 3-weeks following administration as a single dose (e.g., 2×10¹¹ to 2×10¹² vg/kg) to a subject that are increased by about 50% or more (about 75% or more, about 100% or more, about 125% or more, about 150% or more, about 175% or more, or even about 200% or more) as compared to that produced by administration of a non-enveloped viral vector of the same type under the same conditions (e.g., same transgene, same subject, same dose and route of administration, etc., with the only difference being the vector).

Also, in some embodiments, the enveloped viral vector (e.g., enveloped AAV) comprising immunosuppressive molecules in the envelope, as provided herein, provides transgene expression levels 3-weeks following administration as a single dose (e.g., 2×10¹¹ to 2×10¹² vg/kg) to a subject that are increased by about 20% or more (about 50% or more, about 75% or more, about 100% or more, about 125% or more, about 150% or more, about 175% or more, or even about 200% or more) as compared to that produced by administration of an enveloped viral vector of the same type without the immunosuppressive molecules (produced from the same type of producer cell with the exception that the host cell was not engineered to express the immunosuppressive molecules) under the same conditions (e.g., same transgene, same subject, same dose and route of administration, etc., with the only difference being the vector).

It is further believed that the enveloped vector comprising immunosuppressive molecules provided herein minimizes global immunosuppression that results from administration of soluble immunosuppressive molecules (e.g., CTLA4/Ig, abatacept). In some embodiments, the enveloped viral vector (e.g., enveloped AAV) comprising immunosuppressive molecules in the envelope, as provided herein, upon administration in an effective amount to a subject, particularly a human, (e.g., a dose of 2×10¹¹ vg/kg or a dose of 5×10¹¹ vg/kg causes global immunosuppression that is less than that caused by a single administration of 10 mg/kg CTLA4/Ig (or, in some embodiments, 2 mg/kg CTLA4/Ig), as measured within 2 to 3 weeks after administration according to an increase in circulating total anti-IgG antibodies, or an increase in antigen specific antibodies, or activated CD4+ or CD8+ T Cells that are stimulated by antigens other than those derived from the vector administered.

Without wishing to be bound to any particular theory or mechanism of action, it is believed that the enveloped viral vector provided herein evades the effect of the host-immune response to the vector or the viral transgene product, either by suppressing the host-immune response and/or shielding the vector from the effect of the host-generated immune response. For instance, the vector of the invention might reduce the number of vector-neutralizing antibodies produced by the host, or might reduce the effectiveness of those antibodies in neutralizing the virus. Similarly, the vector of the invention might reduce the number of host-produced antibodies to the viral transgene product, or might reduce the effectiveness of those antibodies in inhibiting expression of the transgene product. Also, the vector of the invention might reduce inflammation typically associated with conventional gene therapy vectors, resulting in increased transgene expression.

Thus, in some embodiments, the enveloped viral vector has reduced immunogenicity in a host compared to a native or non-enveloped viral particle or to an enveloped viral particle of the same type but with an envelope that is not engineered to include immunosuppressive molecules in the envelope. In some embodiments, the enveloped viral vector reduces host immunity to the viral transgene product compared to a vector of the same type comprising a native or non-enveloped viral particle or an enveloped viral particle of the same type but with an envelope that is not engineered to include immunosuppressive molecules in the envelope.

### (A) Viral Vectors

Any viral vector that can associate with a lipid bilayer so as to provide an enveloped virus can be used. In some embodiments, the enveloped viral particle is a type that is not typically enveloped in its native state, such as adeno-associated virus (AAV) particles and adenoviral particles. In other embodiments, the native viral particle is of a type that is typically enveloped, such as retroviruses and herpes viruses.

In some embodiments, the viral vector comprises an AAV viral particle. AAV is a member of the parvovirus family and is not typically used as an enveloped virus. Any AAV vector suitable for delivering a transgene can be used. The AAV particle can comprise an AAV capsid protein and an AAV viral genome from any serotype. AAV serotypes include, but are not limited to AAV1, AAV2, AAV3, AAV4, AAV5, AAV6, AAV7, AAV8, AAV9, AAV10, AAV11 or AAV12. In some embodiments, the AAV viral particle comprises an AAV viral capsid and an AAV viral genome from the same serotype. In other embodiments, the AAV viral genome and AAV capsid are of different serotypes. For example, the AAV viral capsid may be an AAV6 viral capsid and the AAV viral genome may be an AAV2 viral genome. In some embodiments, the AAV is a self-complementary AAV (scAAV). In some embodiments, the vector is an AAV8 or AAV2/8 vector, particularly scAAV8 or scAAV2/8).

In some embodiments, the enveloped viral vector comprises lentiviral particles. Any lentivirus suitable for transgene delivery can be used, including but not limited to human immunodeficiency virus, simian immunodeficiency virus and feline immunodeficiency virus. Typically, the lentiviral vector is non-replicating. The lentiviral vector can be an integrating or non-integrating lentiviral vector. In some embodiments, the lentiviral genome lacks vif, vpr, vpu, tat, rev, nef genes. In some embodiments, the lentiviral genome comprises a heterologous transgene, a 5′ long terminal repeat (LTR) and a 3′ LTR, wherein all or part of a U3 region of the 3′ LTR is removed or replaced by a heterologous regulatory element.

The viral particle, specifically the viral genome, will include a heterologous nucleic acid (e.g., a transgene) to be delivered (the “payload”) or can be an empty vector. The particular nature of the nucleic acid to be delivered depends on the desired end-use, and the enveloped vector of the invention is not limited to any particular use or payload. In some embodiments, the payload nucleic acid will express a biological protein, e.g., Factor VIII (e.g., human F8 (UniProtKB-Q2VF45), SQ-FVIII variant of a B-domain-deleted (BDD) human Factor VIII gene (Lind et al., 1995 Eur J Biochem. Aug 15; 232(1):19-27)) or other known variants), Factor IX (e.g., human Factor IX UniProtKB-P00740; or human Factor IX (R338L) “Padua” (Monahan et al., 2015 Hum Gene Ther., 26(2): 69-81, or other known variants), myotubularin, SMN, RPE65, NADH-ubiquinone oxidoreductase chain 4, CHM, huntingtin, alpha-galactosidase A, acid beta-glucosidase, alpha-glucosidase, Ornithine transcarbomylase, argininosuccinate synthetase, β-globin, γ-globin, phenylalanine hydroxylase, or ALD. In some embodiments, the payload nucleic acid sequence encodes the human Factor VIII amino acid sequence of SEQ ID NO.1 or is derived from the amino acid sequence of SEQ ID NO:1. In some embodiments, the payload nucleic acid sequence encodes a human Factor VIII amino acid sequence having more than about any of 80%, 85%, 90%, or 99% identity to the amino acid sequence of SEQ ID NO.1. In some embodiments, the payload nucleic acid sequence encodes the human Factor IX amino acid sequence of SEQ ID NO.1. In some embodiments, the payload nucleic acid sequence encodes a human Factor IX amino acid sequence having more than about any of 80%, 85%, 90%, or 99% identity to the amino acid sequence of SEQ ID NO.2. In other embodiments, the payload nucleic acid encodes a reporter molecule, e.g., green fluorescent protein, red fluorescent protein, yellow fluorescent protein, luciferase, alkaline phosphatase, or beta-galactosidase. In still other embodiments, the payload nucleic acid encodes a therapeutic nucleic acid, such as a siRNA, miRNA, shRNA, antisense RNA, RNAzyme, or DNAzyme. In still other embodiments, the payload nucleic acid encodes one or more gene editing gene products, such as an RNA-guided endonuclease (e.g., Cas9, CPF1, etc.), a guide nucleic acid for an RNA-guided endonuclease, a donor nucleic acid, or some combination thereof.

The heterologous nucleic acid can be under control of a suitable promoter, which can be a tissue specific promoter. For example, if the vector is to be delivered to the liver, a liver-specific promoter (e.g., a liver-specific human al-antitrypsin (hAAT) promoter). Other regulator elements as may be appropriate for a given application also may be included.

(B) Engineered Envelope with Immunosuppressive Molecules

The envelope of the viral vector provided herein comprises a lipid bilayer that partially or completely surrounds the viral particle. Any lipid bilayer can be used, including naturally occurring or synthetic (artificial) lipid bilayers. Synthetic lipid bilayers include, for example, liposomes. Naturally occurring lipid bilayers include any of various types of extracellular vesicles (EVs) known in the art, including exosomes, microvesicles (e.g., shedding vesicles or ectosomes), and the like. For instance, the lipid bilayer of the envelope of the vector can be provided by a portion of a cell membrane that has “budded” from a producer cell, particularly a producer cell that has been engineered to overexpress one or more immunosuppressive molecules as compared to a non-engineered producer cell of the same type. Such a lipid bilayer comprises a portion of a cell membrane from which it is shed. In some embodiments, the lipid bilayer comprises endosome-associated proteins (Alix, Tsg101, and Rab proteins); tetraspanins (CD9, CD63, CD81, CD82, CD53, and CD37); lipid raft-associated proteins (glycosylphosphatidylinositol and flotillin), and/or lipids comprising cholesterol, sphingomyelin, and/or glycerophospholipids. In some embodiments, the lipid bilayer is an exosomal lipid bilayer (e.g., the lipid bilayer is an exosome), particularly the exosomal lipid bilayer of a producer cell (i.e., shed from other otherwise derived from or produced by a producer cell) that is engineered to overexpress one or more immunosuppressive molecules as described herein.

While any cell type can provide EVs, it is sometimes advantageous to avoid the use of tumor cells as producer cells in the context of the invention, due to the potential for contamination by agents (e.g., genetic elements) that contribute to the immortalization of the tumor cell and might be oncogenic or otherwise detrimental to a subject. Thus, in some embodiments, the lipid bilayer is a non-tumor EV lipid bilayer, such as a non-tumor exosomal lipid bilayer (e.g., the lipid bilayer is from a non-tumor EV such as a non-tumor exosome, meaning that the EV or exosome does not have a tumor-cell origin). In other embodiments, the lipid bilayer is an EV lipid bilayer (e.g., an exosomal lipid bilayer or an exosome) from a 293 cell (e.g., HEK293 or HEK293T), particularly an EV lipid bilayer (e.g., an exosomal lipid bilayer or an exosome) a non-tumor producer cell (i.e., shed from other otherwise derived from or produced by a producer cell), such as a 293 cell, that is engineered to overexpress one or more immunosuppressive molecules as described herein.

The envelope also comprises immunosuppressive molecules. The immunosuppressive molecules can be associated with the lipid bilayer of the envelope in any manner. In some embodiments, the immunosuppressive molecule is embedded within or on the lipid bilayer. For instance, the immunosuppressive molecule can comprise, either naturally or synthetically, a transmembrane domain, which integrates into the lipid bilayer. Transmembrane domains are known in the art including but not limited to the PDGR transmembrane domain. Methods of incorporating transmembrane domains (e.g., by generating fusion proteins) are known in the art.

The immunosuppressive molecule can be any molecule that reduces the host immune response to the enveloped vector of the invention as compared to the same vector without the envelope or with an envelope that is not engineered to contain immunosuppressive molecules. The immunosuppressive molecules include but are not limited to molecules (e.g., proteins) that down-regulate immune function of a host by any mechanism, such as by stimulating or up-regulating immune inhibitors or by inhibiting or down-regulating immune stimulating molecules and/or activators, or by otherwise reducing the immunogenicity of the enveloped viral vector compared to an enveloped vector without the immunosuppressive molecules. Immunosuppressive molecules include, but are not limited immune checkpoint receptors and ligands. Non-limiting examples of immunosuppressive molecules include, for instance, CTLA-4 and its ligands (e.g., B7-1 and B7-2), PD-1 and its ligands (e.g., PDL-1 and PDL-2), VISTA, TIM-3 and its ligand (e.g., GAL9), TIGIT and its ligand (e.g., CD155), LAG3, VISTA, and BTLA and its ligand (e.g., HVEM). Also included are active fragments and derivatives of any of the foregoing checkpoint molecules; agonists of any of the foregoing checkpoint molecules, such as agonistic antibodies to any of the foregoing checkpoint molecules; antibodies that block immune stimulatory receptors (co-stimulatory receptors) or their ligands, such as anti-CD28 antibodies; or peptides that mimic the immune functions of immune checkpoint molecules. To the extent a desired immunosuppressive molecule does not natively include a transmembrane domain, the immunosuppressive molecules can be engineered to embed in a lipid bilayer by creating chimeric molecules comprising an extracellular domain, a transmembrane domain, and, optionally, either full length intracellular domains, or any minimal intercellular domain that may be necessary to maintain chimeric molecule expression and binding to its ligand or receptor; as illustrated in FIG. 2. The transmembrane domains and intercellular domains of effector molecules can comprise immunoglobulin Fc receptor domains (or transmembrane region thereof) or any other functional domain necessary to maintain expression and ligand binding activities.

The envelope can comprise any one or more different types of immunosuppressive molecules; however, in some embodiments, the envelope comprises a combination of two or more different immunosuppressive molecules (e.g., three or more different immunosuppressive molecules, four or more different immunosuppressive molecules, or even five or more different immunosuppressive molecules). Thus, for example, in some embodiments, the envelope comprises a combination of two or more different immune checkpoint molecules (e.g., three or more different immune checkpoint molecules, four or more different immune checkpoint molecules, or even five or more different immune checkpoint molecules), optionally two or more (e.g., three or more, four or more, or even five or more) molecules selected from CTLA-4 and its ligands (e.g., B7-1 and B7-2), PD-1 and its ligands (e.g., PDL-1 and PDL-2), VISTA, TIM-3 and its ligand (e.g., GAL9), TIGIT and its ligand (e.g., CD155), LAG3, VISTA, and BTLA and its ligand (e.g., HVEM); active fragments and derivatives of any of the foregoing checkpoint molecules; agonists of any of the foregoing checkpoint molecules, such as agonistic antibodies to any of the foregoing checkpoint molecules; antibodies that block immune stimulatory receptors (co-stimulatory receptors) or their ligands, such as anti-CD28 antibodies; or peptides that mimic the immune functions of immune checkpoint molecules. In some embodiments the envelope comprises CTLA-4 and PD-L1 and PD-L2 and VISTA, or any combination of these, or other immune suppressing molecules, singly or in combinations of up to 4 different molecules. In some embodiments, the envelope comprises CTLA-4 and PD-L1, CTLA-4 and PD-L2, CTLA-4 and PD-1, CTLA-4 and VISTA, CTLA-4 and anti-CD28, PD-1 and VISTA, B7-1 and PD-L1, B7-1 and PD-L2, B7-land PD-1, B7-1 and VISTA, B7-1 and anti-CD28, B7-2 and PD-L1, B7-2 and PD-L2, B7-2and PD-1, B7-2 and VISTA, B7-2 and anti-CD28, PD-1 and VISTA, PD-1 and anti-CD-28, VISTA and anti-CD28, PD-L1 and VISTA, PD-L1 and anti-CD-28, PD-L2 and VISTA, PD-L2 and anti-CD-28, or VISTA and anti-CD28. In some embodiments, the envelope comprises CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-L1 and VISTA. In some embodiments, the immunosuppressive molecules include, or are engineered to include, a transmembrane domain. The immunosuppressive molecule used in the vector should be that of the species of mammal to which the vector is to be administered. Thus, for use in humans, the human ortholog of the immunosuppressive molecule should be used, which proteins are well-known in the field. In a particular embodiment, the immunosuppressive molecules included in the envelope comprise, consist essentially of, or consist of, CTLA-4 and PD-L1. Human CTLA-4 is provided, for instance, by the protein identified by NCBI Reference Sequence: NP_005205.2, and PD-L1 is provided, for instance, by the protein identified by NCBI Reference Sequence: NP_054862.1. In some embodiments, the immunosuppressive molecule is (or derived from) a CTLA-4 molecule comprising the amino acid sequence of SEQ ID NO:3. In some embodiments, the immunosuppressive molecule is (or derived from) a CTLA-4 molecule comprising an amino acid sequence having more than about any of 80%, 85%, 90%, or 99% identity to the amino acid sequence of SEQ ID NO.3. In some embodiments, the immunosuppressive molecule is (or derived from) a PDL-1 molecule comprising the amino acid sequence of SEQ ID NO:4. In some embodiments, the immunosuppressive molecule is (or derived from) a PDL-1 molecule comprising an amino acid sequence having more than about any of 80%, 85%, 90%, or 99% identity to the amino acid sequence of SEQ ID NO.4.

The envelope can comprise the immunosuppressive molecules in any suitable amount or concentration. In some embodiments, the envelope comprises the immunosuppressive molecules in an amount sufficient to improve delivery and expression of the transgene as compared to the same enveloped vector that is not engineered to contain the immunosuppressive molecules. As explained in greater detail in connection with the method of producing the enveloped vectors, the enveloped vector comprising sufficient concentration of immunosuppressive molecules in the envelope can be provided by engineering the host (producer) cell to overexpress the immunosuppressive molecules as compared to the native host cell. Thus, in some embodiments, the envelope of the vector provided herein comprises one or more (or all) of the immunosuppressive molecules in an amount greater than the same enveloped vector produced from the same host cell that has not been engineered to overexpress the immunosuppressive molecules. For instance, the envelope of the vector provided herein, in some embodiments, comprises one or more (or all) of the immunosuppressive molecules in an amount greater than the same enveloped vector produced from the same host cell that has not been engineered to overexpress the immunosuppressive molecules by about 2× or more, by about 3× or more, by about 5× or more, by about 10× or more, by about 20× or more, by about 50× or more, or even about 100× or more (e.g., about 1000× or more). In some embodiments, the host cell is engineered to overexpress one or more (or all) of the immunosuppressive molecules by about 2× or more, about 3× or more, about 5× or more, about 10× or more, about 20× or more, about 50× or more, or even about 100× or more (e.g., about 1000× or more) than the same host cell that is not engineered to overexpress the immunosuppressive molecules. As explained above, in some embodiments, the host cell is a non-tumor host cell engineered to overexpress the immunosuppressive molecules, and the envelope comprises a non-tumor EV lipid bilayer, such as a non-tumor exosomal lipid bilayer, from a non-tumor cell engineered to overexpress the immunosuppressive molecules. In a particular embodiments, the lipid bilayer is an EV lipid bilayer (e.g., an exosomal lipid bilayer or an exosome) from a 293 cell (e.g., HEK293 or any variation thereof, such as HEK293E, HEK293F, HEK293T, etc.) engineered to overexpress the immunosuppressive molecules. The amount of immunosuppressive molecules on the surface of vectors (e.g., in the vector envelope) can be determined using any of various techniques known in the art. For instance, ELISA can be used to measure the amount of such molecules on the surface of vectors and determine the relative amounts of such molecules on different vectors.

The enveloped viral vector provided herein can have any suitable particle size. Typically, the enveloped viral particles will have a size in the range of about 30-600 nm, such as about 50-300 nm, with an average particle size in the range of about 75-150 nm, such as about 80-120 nm (e.g., about 90-115 nm) as measured using a NANOSIGHT™ NS300 (Malvern Instruments, Malvern, United Kingdom) following the manufacturer's protocol. The enveloped viral vectors can each comprise a single capsid or multiple capsids within a single envelope.

### (C) Other Envelope Moieties

The enveloped viral vector provided herein can further include additional moieties in the envelope as desired to provide different functions. For instance, the envelope can be engineered to contain membrane surface proteins that target the vector to a desired cell or tissue type, for instance, a molecule that specifically binds to a ligand or receptor on a desired cell type. In some viral vectors, such as AAV, cell or tissue specificity of the vector can be determined, at least in part, by the serotype of the virus. By engineering the vectors provided herein to contain envelope-bound targeting moieties (e.g. targeting proteins) that bind to ligands or receptors on a desired cell type, the vectors enable more precise targeting as well as options for targeting a wider selection of cell types as compared to relying on AAV serotype specificity alone. For example, to treat Hemophilia B using human Factor IX protein, the envelope of the vector can be engineered to include a moiety that specifically or preferentially binds a surface protein expressed specifically or preferentially on liver cells (e.g., a protein, such as a membrane-bound antigen binding domain (e.g., domain of clone 8D7, BD Biosciences), that specifically binds asialoglycoprotein receptor 1(ASGR1)). Other targeting molecules that target different cell or tissue types can be used depending on the desired destination for the vector. Non-limiting examples include one or more of liver, muscle, heart, brain (e.g., neurons, glial cells, astrocytes, etc.), kidney, lung, pancreas, stomach, intestines, bone marrow, blood cells (e.g., leukocytes, lymphocytes, erythrocytes), ovaries, uterus, testes, or stem cells of any type. As explained in greater detail in connection with the method of producing the vectors, such a vector envelope can be provided by engineering host cells (producer cells) to express high levels of a membrane bound targeting moiety. Thus, in some embodiments, the invention provides a viral vector comprising an envelope wherein the envelope comprises an immunosuppressive molecule and a targeting molecule.

The enveloped viral vector can further comprise additional elements that improve effectiveness or efficiency of the vector, or improve production. For example, exogenous expression of Tetraspanin CD9 in producer cells can improve vector production without degrading vector performance (Shiller et al., Mol Ther Methods clin Dev, (2018) 9:278-287). Thus, the vector might include CD9 in the envelope. However, in some embodiments, the enveloped viral vector is substantially or completely free of elements that significantly impair the efficiency or effectiveness of the vector in delivering nucleic acid to a subject, render the vector unsuitable for use in humans (e.g., under FDA regulations), or substantially impair vector production.

### IV. APPLICATIONS AND METHODS OF USE

The enveloped viral vectors provided herein are useful for the delivery and expression of a nucleic acid (transgene) to a cell or subject. Thus, the invention provides a method of delivering a nucleic acid (transgene) to a cell or subject by administering the enveloped viral vector to the cell or subject.

In some embodiments, the enveloped viral vector, which comprises immunosuppressive molecules in the envelope, can deliver the nucleic acid (transgene) to the cell or subject more effectively or efficiently than a non-enveloped viral vector of the same type or an enveloped viral vector of the same type but without an envelope engineered to comprise the immunosuppressive molecules. In some embodiments, the more effective or efficient delivery results in a higher viral genome copy per target cell, and/or higher expression of the transgene product (as applicable) in the cell or subject. For instance, in some embodiments, the enveloped viral vector (e.g., enveloped AAV) comprising immunosuppressive molecules in the envelope, as provided herein, provides transgene expression levels 3-weeks following administration to a subject that are increased by about 50% or more (about 75% or more, about 100% or more, about 125% or more, about 150% or more, about 175% or more, or even about 200% or more) as compared to that produced by administration of a non-enveloped viral vector of the same type under the same conditions (e.g., same transgene, same subject, same dose and route of administration, etc., with the only difference being the vector). Also, in some embodiments, the enveloped viral vector (e.g., enveloped AAV) comprising immunosuppressive molecules in the envelope, as provided herein, provides transgene expression levels 3-weeks following administration to a subject that are increased by about 20% or more (about 50% or more, about 75% or more, about 100% or more, about 125% or more, about 150% or more, about 175% or more, or even about 200% or more) as compared to that produced by administration of an enveloped viral vector of the same type without the immunosuppressive molecules (produced from the same type of producer cell with the exception that the host cell was not engineered to express the immunosuppressive molecules) under the same conditions (e.g., same transgene, same subject, same dose and route of administration, etc., with the only difference being the vector).

In addition, or alternatively, some embodiments of the enveloped viral vector comprising immunosuppressive molecules are believed to reduce the host immune response to the vector or transgene product, or the impact of the host immune response on transgene delivery and/or expression. Thus, in some embodiments, the enveloped viral vector provided herein allows for repeat dosing of the vector and/or dosing of subjects with pre-existing immunity to a given virus type (e.g., AAV of a particular serotype). Accordingly, in one aspect, the method comprises administration of the enveloped viral vector to a subject previously exposed to a virus of the same type contained in the enveloped viral vector (either by natural exposure to the native virus or by prior administration of the viral vector), or a subject that otherwise has a pre-existing immunity to the virus (e.g., a patient that has pre-existing antibodies to the virus). Thus, the method can comprise administering the enveloped viral vector to the subject in a repeat dosing schedule comprising two or more separate administrations of a dose of a the enveloped viral vector separated by a suitable time interval (e.g., two or more administrations of a dose of the enveloped viral vector separated by at least a day, at least a week, at least two weeks, at least three weeks, at least four weeks or a month, at least two months, at least three months, at least six months, or even at least a year or more).

Although the vector comprises immunosuppressive molecules, the total amount of the immunosuppressive molecule in a dose of the vector will typically be less than the dose of the immunosuppressive molecule that would be used when administered as a soluble immunosuppressive agent. Thus, for instance, in CTLA4/Ig might be used as an immunosuppressive agent at a dose of 10 mg/kg. However, in some embodiments, a single dose of vector (e.g., 2×10¹¹ vg/kg or even 5×10¹¹ vg/kg) will have far less of the immunosuppressive agent (e.g., membrane-bound CTLA4), such as less than about 5 mg/kg, less than about 2 mg/kg, less than about 1 mg/kg, or even less than about 0.5 mg/kg (e.g., less than about 0.1 mg/kg). Accordingly, in some embodiments, the enveloped vector comprising immunosuppressive molecules provided herein minimizes global immunosuppression that results from administration of soluble immunosuppressive agents (e.g., CTLA4/Ig, abatacept). In some embodiments, the enveloped viral vector (e.g., enveloped AAV) comprising immunosuppressive molecules in the envelope, as provided herein, upon administration in an effective amount to a subject, particularly a human, (e.g., a dose of 2×10¹¹ vg/kg or a dose of 5×10¹¹ vg/kg causes global immunosuppression that is less than that caused by a single administration of 10 mg/kg CTLA4/Ig (or, in some embodiments, 2 mg/kg CTLA4/Ig), as measured within 2-3 weeks after administration according to an increase in circulating total anti-IgG antibodies, or an increase in antigen specific antibodies, or activated CD4+ or CD8+ T Cells that are stimulated by antigens other than those derived from the vector administered.

The enveloped viral vector can be administered to deliver a nucleic acid (transgene) to a cell or subject for any ultimate end purpose. In some embodiments, this end purpose might be to express the transgene in a cell in vitro for research purposes, or for the production of a protein or other bio-production process. In other embodiments, the enveloped viral vector is used to treat a disease or disorder in an individual. The disease or disorder can be any disease or disorder susceptible to treatment by delivery and (if applicable) expression of a nucleic acid or transgene. In some embodiments, the disease or disorder is a monogenic disease. In some embodiments, the disease or disorder is a lysosomal storage disease. In some embodiments, the disease or disorder is a glycogen storage disease. In some embodiments, the disease or disorder is a hemoglobin disorder. In some embodiments, the disease or disorder is a musculoskeletal disorder. In some embodiments, the disease or disorder is a CNS disease or disorder. In some embodiments, the disease or disorder is a cardiovascular disorder including heart disease or stroke. In some embodiments, the disease is a cancer.

More specific illustrative, but non-limiting, examples of diseases include myotobularin myopathy, spinal muscular atrophy, Leber congenital amaurosis, hemophilia A and B, Niemann Pick disease (e.g., Niemann Pick A, Niemann Pick B, Niemann Pick C), choroideremia, Huntington's disease, Batten disease, Leber hereditary optic neuropathy, ornithine transcarbamylase (OTC) deficiency, glycogen storage diseases, Pompe disease, Wilson disease, citrullinemia Type 1, PKU (phenylketonuria), adrenoleukodystrophy, hemoglobin disorders including sickle cell disease, beta thalassemia, central nervous system disorders, and musculoskeletal disorders. Thus, in some embodiments of the method, the enveloped viral vector is administered to a subject that has such a disease or disorder or is at risk of developing the disease or disorder (e.g. carries a mutation for the disease or disorder or has a family history of the disease or disorder). Furthermore, when used to treat a disease or disorder, the enveloped viral vector comprises a payload nucleic acid the expression of which treats the disease of the subject. By way of non-limiting example, the nucleic acid might encode one or more of the following: Factor VIII, Factor IX, myotubularin, SMN, RPE65, NADH-ubiquinone oxidoreductase chain 4, CHM, huntingtin, alpha-galactosidase A, acid beta-glucosidase, alpha-glucosidase, Ornithine transcarbomylase, argininosuccinate synthetase, β-globin, γ-globin, phenylalanine hydroxylase, or ALD.

The method also can be used to deliver a therapeutic nucleic acid to a cell or subject for the treatment of disease or any other purpose. In some embodiments, the therapeutic nucleic acid is a siRNA, miRNA, shRNA, antisense RNA, RNAzyme, or DNAzyme.

Also provided is a method of using the enveloped viral vector for delivering a nucleic acid encoding one or more gene editing gene products to a cell in vitro or in vivo. In some embodiments, the one or more gene editing gene products is an RNA-guided endonuclease (e.g., Cas9 or Cpf1), one or more guide sequences for the RNA-guided endonuclease, and/or one or more donor sequences.

In any of the foregoing methods, the cell may be any type of cell, particularly a mammalian cell or human cell. The subject can be any subject, such as a human, a non-human primate, or other mammal including a rodent (e.g., a mouse, a rat, a guinea pig, a hamster), a rabbit, a dog, a cat, a horse, a cow, a pig, a sheep, a frog, or a bird.

In any of the foregoing methods of treatment, a therapeutically effective amount of the enveloped viral vector is administered to the subject by any suitable route of administration. The effective dose and route of administration will depend upon the indication, and can be determined by the practitioner. In some embodiments, the enveloped viral vector is delivered systemically; for example, intravenously, intra-arterially, intraperitoneally, subcutaneously, orally, or by inhalation. In other embodiments, the enveloped viral vector is delivered directly to a tissue (e.g., an organ, a tumor, etc.), or is administered to the CNS (e.g., intrathecally, to the spinal cord, to a specific part of the brain such as a ventricle, the hypothalamus, the pituitary, the cerebrum, the cerebellum, etc.).

The enveloped viral vector can be used as part of a composition comprising the enveloped viral vector and an appropriate carrier, such as a pharmaceutically acceptable carrier such as saline. Suitable carriers, formulation buffers, and other excipients for formulation of viral vector compositions are known in the art and applicable to the presently provided composition.

In a particular embodiment, a method of treating hemophilia B is provided, which method comprises administering to a subject in need of treatment the enveloped viral vector provided herein, wherein the heterologous transgene encodes a human Factor IX (FIX) protein (e.g., human Factor IX UniProtKB-P00740; human Factor IX (R338L) “Padua” (Monahan et al., (2015) Hum Gene Ther., 26(2):69-81, or other known variants), and wherein the envelope of the viral vector is an engineered lipid bilayer comprising CTLA-4 and PD-L1. In a more particular embodiment, the viral vector is AAV (e.g., AAV8 or AAV2/8, or scAAV8 or scAAV2/8), optionally wherein the envelope is provided by an exosome engineered to contain CTLA-4 and PD-L1 (e.g., an exosome from a producer cell (e.g., an HEK293 cell) engineered to overexpress CTLA-4 and PD-L1). In some embodiments, the human Factor IX comprises the amino acid sequence of SEQ ID NO.1. In some embodiments, the human Factor IX comprises an amino acid sequence having more than about any of 80%, 85%, 90%, or 99% identity to the amino acid sequence of SEQ ID NO.2. In some embodiments, CTLA-4 comprises or is derived from a CTLA comprising the amino acid sequence of SEQ ID NO:3. In some embodiments, the CTLA-4 comprises an amino acid sequence having more than about any of 80%, 85%, 90%, or 99% identity to the amino acid sequence of SEQ ID NO.3. In some embodiments, the PDL-1 comprises or is derived from a PDL-1 comprising the amino acid sequence of SEQ ID NO:4. In some embodiments, the PDL-1 comprises an amino acid sequence having more than about any of 80%, 85%, 90%, or 99% identity to the amino acid sequence of SEQ ID NO.4. In some embodiments, the enveloped viral vector is delivered to the liver, and the heterologous transgene includes a liver-specific promoter. In some embodiments, the vector is administered intravenously, optionally to the hepatic artery. In some embodiments, the vector will be administered in a dose of 2×10¹¹ to 2×10¹² vector genomes (vg) per kilogram bodyweight of the subject (e.g., 2×10¹¹ to 8×10¹¹ or 3×10¹¹ to 6×10¹¹ vector genomes (vg) per kilogram bodyweight of the subject). In some embodiments, the method comprises administering 2 or more doses (e.g., 3 or more doses, 4 or more doses, or 5 or more doses) with an interval of at least one day (at least a day, at least a week, at least two weeks, at least three weeks, at least four weeks or a month, at least two months, at least three months, at least six months, or even at least a year or more) between the doses.

In another particular embodiment, a method of treating hemophilia A is provided, which method comprises administering to a subject in need of treatment the enveloped viral vector provided herein, wherein the heterologous transgene encodes a human Factor VIII (e.g., human F8 (UniProtKB-Q2VF45), SQ-FVIII variant of a B-domain-deleted (BDD) human F8 gene (Lind et al., (1995) Eur J Biochem. August 15; 232(1):19-27), or other known variant), and wherein the envelope of the viral vector is an engineered lipid bilayer comprising CTLA-4 and PD-L1. In a more particular embodiment, the viral vector is AAV (e.g., AAV8 or scAAV8, or scAAV8 or scAAV2/8), optionally wherein the envelope is provided by an exosome produced from a host cell (e.g., an HEK293 cell) engineered to overexpress CTLA-4 and PD-L1. In some embodiments, the human Factor VIII comprises the amino acid sequence of SEQ ID NO.1 or is derived from the amino acid sequence of SEQ ID NO:1. In some embodiments, the human Factor VIII comprises an amino acid sequence having more than about any of 80%, 85%, 90%, or 99% identity to the amino acid sequence of SEQ ID NO.1. In some embodiments, CTLA-4 comprises or is derived from a CTLA comprising the amino acid sequence of SEQ ID NO:3. In some embodiments, the CTLA-4 comprises an amino acid sequence having more than about any of 80%, 85%, 90%, or 99% identity to the amino acid sequence of SEQ ID NO.3. In some embodiments, the PDL-1 comprises or is derived from a PDL-1 comprising the amino acid sequence of SEQ ID NO:4. In some embodiments, the PDL-1 comprises an amino acid sequence having more than about any of 80%, 85%, 90%, or 99% identity to the amino acid sequence of SEQ ID NO.4. In some embodiments, the enveloped viral vector is delivered to the liver, and the heterologous transgene includes a liver-specific promoter. In some embodiments, the vector is administered intravenously, optionally to the hepatic artery. In some embodiments, the vector will be administered in a dose of 2×10¹¹ to 2×10¹² vector genomes (vg) per kilogram bodyweight of the subject (e.g., 2×10¹¹ to 8×10¹¹ or 3×10¹¹ to 6×10¹¹ vector genomes (vg) per kilogram bodyweight of the subject). In some embodiments, the method comprises administering 2 or more doses (e.g., 3 or more doses, 4 or more doses, or 5 or more doses) with an interval of at least one day (at least a day, at least a week, at least two weeks, at least three weeks, at least four weeks or a month, at least two months, at least three months, at least six months, or even at least a year or more) between the doses.

### VI. MANUFACTURING

The enveloped viral vector provided herein can be produced by any suitable method. A non-limiting example is provided by US 2013/020559, incorporated herein by reference.

One particularly advantageous method involves producing the enveloped vectors from a producer cell line that has been engineered to overexpress the immunosuppressive molecules desired to be included in the envelope of the vector. Thus, provided herein is a method of preparing an enveloped viral vector with an envelope comprising immunosuppressive molecules, as described herein, by (a) culturing viral producer cells under conditions to generate enveloped viral particles, wherein the viral producer cells comprise a nucleic acid encoding one or more one or more membrane-bound immunosuppressive molecules, and (b) collecting the enveloped viral vectors.

### (A) Producer Cell Engineering

Any producer cell suitable for the conventional production of the virus to be used in the viral vector can be used to produce the enveloped viral vector of the invention. Suitable producer cells include, but are not limited to, 293 cells (e.g., HEK293, HEK293E, HEK293F, HEK293T, and the like), and Hela cells. The producer cells can be engineered to express the desired immunosuppressive molecules by any suitable method. In some embodiments of the invention, immunosuppressive molecules are expressed by transfection, either stably or transiently, of an exogenous nucleic acid (e.g., plasmids or other vectors) encoding the immunosuppressive molecules into producer cells. By expression of such exogenous nucleic acids, the producer cells overexpress the immunosuppressive molecules as compared to the same producer cell that has not been transfected with exogenous nucleic acids encoding the immunosuppressive molecules, and enveloped virus that buds from the producer cell, in turn, has increased amounts of the immunosuppressive molecules as compared to an enveloped virus budding from the same producer cell that has not been engineered to overexpress the immunosuppressive molecules. In some embodiments, the host cell that is engineered to overexpress the immunosuppressive molecules by about 2× or more, about 3× or more, about 5× or more, about 10× or more, about 20× or more, about 50× or more, or even about 100× or more than the same host cell that is not engineered to overexpress the immunosuppressive molecules.

Expression of the immunosuppressive molecules can be driven by a promoter, such as a constitutive promoter (e.g., a CMV promoter). In some embodiments, the gene encoding the effector molecule is followed by polyadenylation signal (e.g., a hemoglobin polyadenylation signal) downstream of the effector molecule coding region. In some embodiments, an intron is inserted downstream of the promoter. For example, a hemoglobin derived artificial intron downstream of the promoter may be employed to increase effector molecule production. The method for transient transfections includes but is not limited to calcium phosphate transfection. The method to produce stable cell lines expressing single or combined immune modulators includes but is not limited to retroviral gene transfer or concatemer transfection followed by selection (Throm et al. (2009) Blood, 113(21): 5104-5110). The producer cells are engineered in this way to express individual immunosuppressive molecules, or to express different combinations of immunosuppressive molecules, as may be desired in the enveloped vector. The producer cells also can be engineered in other ways known in the art to increase productivity. For example, the producer cells can be engineered to overexpress Tetraspanin CD9 to improve vector production (Shiller et al., (2018) Mol Ther Methods Clin Dev, 9:278-287).

### (B) Production of Enveloped Viral Vectors

The enveloped vectors described herein can be produced from the engineered producer cells by any suitable technique. The particular technique used will depend upon the type of virus used in the enveloped viral vector. For example, enveloped AAV vectors can be produced by co-transfecting plasmids or other expression vectors encoding the viral production genes (e.g., Rep/Cap and helper genes) and a plasmid or other construct comprising the AAV ITR and payload nucleic acid. Transfection can be accomplished in any manner, such as by using calcium phosphate transfection, polyethyleneimine (PEI) transfection, or by using an HSV based production system (Booth et al. (2004) Gene Ther, 11(10):829-837). In the case of AAV, the viral genes can include but are not limited to AAV2, 5, 6, 8, or 9 structural genes Rep and Cap, flanked by the AAV2 ITRs, and necessary helper virus genes (Ayuso et al. (2014) Hum Gene Ther, 25:977-987). Production can be done in any suitable manner, such as by using an adherent or suspension production system, with or without serum (Ayuso et al. (2014) Hum Gene Ther, 25:977-987; Xiao et al. (1998), J Virol, 72(3): 2224-2232; Ryu et al. (2013) Mol Ther, Volume 21.B, which methods may optionally include the following modification: prior to cesium chloride or iodixanol gradient purification, clarified harvested supernatant will be used on an affinity purification column to enrich for enveloped virus). When the enveloped viral vector includes a targeting moiety as described herein, the targeting moiety can be used as an affinity ligand to aid in isolation/purification. Other methods for producing enveloped AAV vectors are known and can be used, as are methods for producing enveloped viruses of different types (e.g., enveloped lentivirus), provided the producer cell is engineered to overexpress the desired immunosuppressive molecules. In the case of lentivirus-based vectors, necessary viral genes are supplied by co-transfecting of multiple plasmids, using a similar purification method.

Vectors are harvested after an empirically determined length of time, and then purified using any of various techniques known in the art, provided the purification used does not remove the envelope from the virus. Purifications techniques can include but are not limited to ion-exchange chromatography, size exclusion chromatography, affinity chromatography, and tangential flow filtration. Ultracentrifugation, including continuous ultracentrifugation, may be used to purify the enveloped viral vectors.

The amounts of enveloped viral vectors produced per liter of producer cells can be increased using various methods. These methods can include but are not limited to adding molecules that suppress apoptosis, or suspend cell division to the producer cell during fermentation. Molecules or compounds that alter the lipid composition of producer cell membranes may also be used to increase vector production per liter. Additionally, compounds or molecules that increase exosome production, including membrane fusigenic molecules.

Thus, in some embodiments, the invention provides a method of producing an enveloped viral vector as described herein, the method comprising (a) culturing viral producer cells under conditions to generate enveloped viral particles, wherein the viral producer cells comprise nucleic acids encoding one or more one or more membrane bound immunosuppressive molecules, and (b) collecting the enveloped viral vectors. The enveloped viral vector can have any of the features and elements described herein with respect to the enveloped viral vector of the invention. Furthermore, the producer cells can have any of the features and elements described in the previous sections, and the method of producing the enveloped viral vector can further include steps of providing the producer cells by, for instance, transforming the producer cells with nucleic acids encoding the one or more membrane-bound immunosuppressive molecules. In some embodiments, the host cell is engineered to overexpress the immunosuppressive molecules (e.g., comprises one or more exogenous nucleic acids encoding the immunosuppressive molecules) by about 2× or more, about 3× or more, about 5× or more, about 10× or more, about 20× or more, about 50× or more, or even about 100× or more than the same host cell that is not engineered to overexpress the immunosuppressive molecules. In some embodiments, the host cell is a non-tumor cell, such as a 293 cell (e.g., HEK293, HEK293T, HEK293E, HEK293F, etc.).

Collection of the enveloped viral vector can comprise isolating the enveloped virus from the culture fluid of the cultured viral producer cells. Collection can be performed by any method that does not remove the envelope from the virus. Thus, for instance, the collection can comprise separation of the enveloped virus from the cell culture by ultracentrifugation or other suitable method. The method preferably avoids the use of detergents. Furthermore, the method preferably minimizes or avoids lysis of the producer cells prior to collection of the enveloped virus, as the lysis of the producer cells will release non-enveloped virus into the culture.

In some embodiments, the enveloped viral vector is an enveloped AAV vector and the viral producer cells comprise (i) a nucleic acid encoding AAV rep and cap genes, (ii) a nucleic acid encoding an AAV viral genome comprising a transgene and at least one ITR, and (iii) a nucleic acid encoding AAV helper genes. In some embodiments, nucleic acid encoding AAV rep and cap genes and/or the AAV viral genome is transiently introduced in the producer cell line. In some embodiments, nucleic acid encoding AAV rep and cap genes and/or the AAV viral genome is stably maintained in the producer cell line. In some embodiments nucleic acid encoding AAV rep and cap genes and/or the AAV viral genome is stably integrated into the genome of the producer cell line. In some embodiments, the AAV genome comprises two AAV ITRs (e.g., the viral genome comprises a heterologous transgene flanked by AAV ITRs). In some embodiments, one or more AAV helper functions are provided by one or more of a plasmid, an adenovirus, a nucleic acid stably integrated into the cell genome or a herpes simplex virus (HSV). In some embodiments, the AAV helper functions comprise one or more of adenovirus E1A function, adenovirus E1B function, adenovirus E2A function, adenovirus E4 function and adenovirus VA function. In some embodiments, one or more AAV helper functions are stably integrated into the host cell genome and other AAV helper functions are delivered transiently. For example, in some embodiments, the AAV enveloped vector is prepared in 293 cells expressing adenovirus E1A and E1B functions. The other helper functions are delivered transiently; for example, by plasmid or by replication-deficient adenovirus. In some embodiments, the AAV helper functions comprise one or more of HSV UL5 function, HSV UL8 function, HSV UL52 function, and HSV UL29 function.

In some embodiments, the invention provides a method of producing an enveloped lentiviral vector as described herein, the method comprising (a) culturing viral producer cells under conditions to generate enveloped viral particles, wherein the viral producer cells comprise nucleic acid encoding one or more one or more membrane bound immunosuppressive molecules, and (b) collecting the enveloped lentiviral vectors. In some embodiments, the lentiviral vector is a human immunodeficiency virus, a simian immunodeficiency virus or a feline immunodeficiency virus. In some embodiments, the viral producer cells comprise (a) nucleic acid encoding lentiviral gag gene, (b) nucleic acid encoding lentiviral pol gene, (c) nucleic acid encoding a lentiviral transfer vector comprising a transgene, a 5′ long terminal repeat (LTR) and a 3′ LTR, wherein all or part of a U3 region of the 3′ LTR is replaced by a heterologous regulatory element or as described (Ryu et al. (2013) Mol Ther 2013, Volume 21.B.; Meliani et al. (2015) Hum Gene Ther Methods, 26:45-53).

### VI. KITS

The present invention also provides kits for administering the enveloped viral vectors described herein to a cell or subject according to the methods of the invention. The kits may comprise any enveloped viral vector of the invention. For example, the kits may include enveloped AAV vectors or enveloped lentiviral vectors as described herein.

In some embodiments, the kits further include instructions for effector vector delivery. The kits described herein may further include other materials desirable from a commercial and user standpoint, including other buffers, diluents, filters, needles, syringes, and package inserts with instructions for performing any methods described herein. Suitable packaging materials may also be included and may be any packaging materials known in the art, including, for example, vials (such as sealed vials), vessels, ampules, bottles, jars, flexible packaging (e.g., sealed Mylar or plastic bags), and the like. These articles of manufacture may further be sterilized and/or sealed. In some embodiments, the kits comprise instructions for treating a disease disorder described herein using any of the methods and/or effector vectors described herein. The kits may include a pharmaceutically acceptable carrier suitable for injection into the individual, and one or more of: a buffer, a diluent, a filter, a needle, a syringe, and a package insert with instructions for performing injections into the mammal.

In some embodiments, the kits further contain one or more of the buffers and/or pharmaceutically acceptable excipients described herein (e.g., as described in REMINGTON'S PHARMACEUTICAL SCIENCES (Mack Pub. Co., N.J. 1991). In some embodiments, the kits include one or more pharmaceutically acceptable excipients, carriers, solutions, and/or additional ingredients described herein. The kits described herein can be packaged in single unit dosages or in multidosage forms. The contents of the kits are generally formulated as sterile and can be lyophilized or provided as a substantially isotonic solution.

### EXEMPLARY EMBODIMENTS

The following embodiments are provided merely for the purposes of further illustrating the compositions and methods provided herein, and do not limit the invention:

Embodiment 1. A composition comprising an enveloped viral vector, wherein the enveloped viral vector comprises a vector particle surrounded by envelop, wherein the envelope comprises one or more molecules that provide immune effector functions (i.e., immunosuppressive molecules).

Embodiment 2. The composition of embodiment 1, wherein the immune effector functions reduce immunogenicity of the enveloped vector compared to a vector without immune effector molecules.

Embodiment 3. The composition of embodiment 1 or 2, wherein the immune effector functions stimulate immune inhibitors.

Embodiment 4. The composition of embodiment 1 or 2, wherein the immune effector functions inhibit immune stimulating molecules.

Embodiment 5. The composition of any one of embodiments 1-4, wherein envelope comprises molecules that stimulate immune inhibitors and molecules that inhibit immune stimulating molecules.

Embodiment 6. The composition of any one of embodiments 1-5, wherein the one or more molecules providing immune effector functions includes one or more of CTLA4, B7-1, B7-2, PD-1, PD-L1, PD-L2, CD28, VISTA TIM-3, GALS, TIGIT, CD155, LAG3, VISTA, BTLA or HVEM.

Embodiment 7. The composition of any one of embodiments 1-6, wherein the envelope comprises CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-L1 and VISTA.

Embodiment 8. The composition of any one of embodiments 1-7, wherein the one or more molecules that provides immune effector functions comprises a transmembrane domain.

Embodiment 9. The composition of any one of embodiments 1-8, wherein the envelope further comprises targeting molecules that target the vector to one or more cell types.

Embodiment 10. The composition of embodiment 9, wherein the targeting molecules confer tissue specificity to the enveloped vector.

Embodiment 11. The composition of embodiment 10, wherein the targeting molecule is an antibody.

Embodiment 12. The composition of embodiment 11, wherein the antibody is antibody 8D7.

Embodiment 13. The composition of any one of embodiments 9-12, wherein the one or more targeting molecules comprises a transmembrane domain.

Embodiment 14. The composition of any one of embodiments 1-13, wherein the viral vector comprises a viral particle.

Embodiment 15. The composition of embodiment 14, wherein the viral particle comprises a viral capsid and a viral genome, or an enveloped capsid and a viral genome, such as a retrovirus.

Embodiment 16. The composition of embodiment 15, wherein the viral genome comprises one or more heterologous transgenes.

Embodiment 17. The composition of embodiment 16, wherein the heterologous transgene encodes a polypeptide.

Embodiment 18. The composition of embodiment 17, wherein the heterologous transgene encodes a therapeutic polypeptide or a reporter polypeptide.

Embodiment 19. The composition of embodiment 18, wherein the therapeutic polypeptide is Factor VIII, Factor IX, myotubularin, SMN, RPE65, NADH-ubiquinone oxidoreductase chain 4, CHM, huntingtin, alpha-galactosidase A, acid beta-glucosidase, alpha-glucosidase, ornithine transcarbamylase, argininosuccinate synthetase, β-globin, γ-globin, phenylalanine hydroxylase, or ALD.

Embodiment 20. The composition of embodiment 16, wherein the heterologous transgene encodes a therapeutic nucleic acid.

Embodiment 21. The composition of embodiment 20, wherein the therapeutic nucleic acid is a siRNA, miRNA, shRNA, antisense RNA, RNAzyme, or DNAzyme.

Embodiment 22. The composition of embodiment 16, wherein the heterologous transgene encodes one or more gene editing gene products.

Embodiment 23. The composition of embodiment 22, wherein the one or more gene editing gene products is a CAS nuclease and/or one or more guide sequences and/or one or more donor sequences.

Embodiment 24. The composition of any one of embodiments 1-23, wherein the viral vector is an adeno-associated viral (AAV) vector or a lentiviral vector.

Embodiment 25. The composition of any one of embodiments 1-24, wherein the viral vector is an adeno-associated viral vector.

Embodiment 26. The composition of embodiment 25, wherein the AAV vector comprises a capsid from human AAV serotype AAV1, AAV2, AAV3, AAV4, AAV5, AAV6, AAV7, AAV8, AAV9, AAV10, AAV11 or AAV12.

Embodiment 27. The composition of embodiment 25 or 26, wherein the AAV vector comprises an AAV viral genome comprising inverted terminal repeat (ITR) sequences from human AAV serotype AAV1, AAV2, AAV3, AAV4, AAV5, AAV6, AAV7, AAV8, AAV9, r AAV10.

Embodiment 28. The composition of embodiment 27, wherein the AAV capsid and the AAV ITR are from the same serotype or from different serotypes.

Embodiment 29. The composition of embodiment 1-24, wherein the viral vector is a lentiviral vector.

Embodiment 30. The composition of embodiment 29, wherein the lentiviral vector is derived from human immunodeficiency virus, a simian immunodeficiency virus or a feline immunodeficiency virus.

Embodiment 31. The composition of embodiment 29 or 30, wherein the lentiviral vector is non-replicating.

Embodiment 32. The composition of any one of embodiments 29-30, wherein the lentiviral vector is non-integrating.

Embodiment 33. A pharmaceutical composition comprising the composition of any one of embodiments 1-32 and one or more pharmaceutically acceptable excipients.

Embodiment 34. A method of delivering a transgene to an individual comprising administering a composition comprising an enveloped viral vector to the individual, wherein the enveloped viral vector comprises a vector particle surrounded by envelop, wherein the envelope comprises one or more molecules that provide immune effector functions and wherein the viral particle comprises a viral genome comprising the transgene.

Embodiment 35. A method of treating an individual with a disease or disorder comprising administering a composition comprising an enveloped viral vector to the individual in need thereof, wherein the enveloped viral vector comprises a vector particle surrounded by envelop, wherein the envelope comprises one or more molecules that provide immune effector functions and wherein the viral particle comprises a viral genome comprising a therapeutic transgene.

Embodiment 36. The method of embodiment 34 or 35, wherein the immune effector functions reduce immunogenicity of the enveloped vector.

Embodiment 37. The composition of any one of embodiments 34-36, wherein the immune effector functions stimulate immune inhibitors.

Embodiment 38. The method of any one of embodiments 34-36, wherein the immune effector functions inhibit immune stimulating molecules.

Embodiment 39. The method of any one of embodiments 34-38, wherein envelope comprises molecules that stimulate immune inhibitors and molecules that inhibit immune stimulating molecules.

Embodiment 40. The method of any one of embodiments 34-39, wherein the one or more molecules providing immune effector functions includes one or more of CTLA4, B7-1, B7-2, PD-1, PD-L1, PD-L2, CD28, VISTA, TIM-3, GALS, TIGIT, CD155, LAG3, VISTA, BTLA or HVEM.

Embodiment 41. The method of any one of embodiments 34-40, wherein the envelope comprises CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-L1 and VISTA.

Embodiment 42. The method of any one of embodiments 34-41, wherein the one or more molecules that provides immune effector functions comprises a transmembrane domain.

Embodiment 43. The method of any one of embodiments 34-42, wherein the envelope further comprises targeting molecules that target the vector to one or more cell types.

Embodiment 44. The method of embodiment 43, wherein the targeting molecules confer tissue specificity to the enveloped vector.

Embodiment 45. The method of embodiment 44, wherein the targeting molecule is an antibody.

Embodiment 46. The method of embodiment 45, wherein the antibody is antibody 8D7.

Embodiment 47. The method of any one of embodiments 43-46, wherein the one or more targeting molecules comprises a transmembrane domain.

Embodiment 48. The method of any one of embodiments 34-47, wherein the heterologous transgene encodes a polypeptide.

Embodiment 49. The method of embodiment 48, wherein the heterologous transgene encodes a therapeutic polypeptide or a reporter polypeptide.

Embodiment 50. The method of embodiment 49, wherein the therapeutic polypeptide is Factor VIII, Factor IX, Factor VIII, Factor IX, myotubularin, SMN, RPE65, NADH-ubiquinone oxidoreductase chain 4, CHM, huntingtin, alpha-galactosidase A, acid beta-glucosidase, alpha-glucosidase, ornithine transcarbamylase, argininosuccinate synthetase, β-globin, γ-globin, phenylalanine hydroxylase, or ALD.

Embodiment 51. The method of any one of embodiments 34-47, wherein the heterologous transgene encodes a therapeutic nucleic acid.

Embodiment 52. The method of embodiment 51, wherein the therapeutic nucleic acid is a siRNA, miRNA, shRNA, antisense RNA, RNAzyme, or DNAzyme.

Embodiment 53. The method of embodiment 52, wherein the heterologous transgene encodes one or more gene editing gene products.

Embodiment 54. The method of any one of embodiments 34-53, wherein the one or more gene editing gene products is a CAS nuclease and/or one or more guide sequences and/or one or more donor sequences.

Embodiment 55. The method of any one of embodiments 34-54, wherein the viral vector is an adeno-associated viral (AAV) vector or a lentiviral vector.

Embodiment 56. The method of any one of embodiments 34-55, wherein the viral vector is an adeno-associated viral vector.

Embodiment 57. The method of embodiment 56, wherein the AAV vector comprises a capsid from human AAV serotype AAV1, AAV2, AAV3, AAV4, AAV5, AAV6, AAV7, AAV8, AAV9, AAV10, AAV11 or AAV12.

Embodiment 58. The method of embodiment 56 or 57, wherein the AAV vector comprises an AAV viral genome comprising inverted terminal repeat (ITR) sequences from human AAV serotype AAV1, AAV2, AAV3, AAV4, AAV5, AAV6, AAV7, AAV8, AAV9, r AAV10.

Embodiment 59. The method of embodiment 58, wherein the AAV capsid and the AAV ITR are from the same serotype or from different serotypes.

Embodiment 60. The method of embodiment 34-55, wherein the viral vector is a lentiviral vector.

Embodiment 61. The method of embodiment 60, wherein the lentiviral vector is derived from human immunodeficiency virus, simian immunodeficiency virus or feline immunodeficiency virus.

Embodiment 62. The method of embodiment 60 or 61, wherein the lentiviral vector is non-replicating.

Embodiment 63. The method of any one of embodiments 60-52, wherein the lentiviral vector is non-integrating.

Embodiment 64. The method of any one of embodiments 34-63, wherein the composition is a pharmaceutical composition comprising enveloped viral vector and one or more pharmaceutically acceptable excipients.

Embodiment 65. The method of any one of embodiments 34-64, wherein the individual is a human.

Embodiment 66. The method of embodiment 35, wherein the disease or disorder is monogenic disease.

Embodiment 67. The method of embodiment 35, wherein the disease or disorder is myotobularin myopathy, spinal muscular atrophy, Leber's congenital amaurosis, hemophilia A, hemophilia B, choroideremia, Huntington's disease, Batten disease, Leber hereditary optic neuropathy, ornithine transcarbamylase (OTC) deficiency, Pompe disease, Fabry disease, citrullinemia type 1, phenylketonuria (PKU), adrenoleukodystrophy, sickle cell disease, or beta thalessemia.

Embodiment 68. A method of producing an enveloped viral vector with reduced immunogenicity, the method comprising a) culturing a viral producer cells under conditions to generate enveloped viral particles, wherein the viral producer cells comprise nucleic acid encoding one or more one or more membrane bound immune effector functions that reduce immunogenicity of the enveloped vector, and b) collecting the enveloped viral vectors.

Embodiment 69. The method of embodiment 68, wherein the immune effector functions reduce immunogenicity of the enveloped vector.

Embodiment 70. The method of embodiment 68 or 69, wherein the immune effector functions stimulate immune inhibitors.

Embodiment 71. The method of embodiment 68 or 69, wherein the immune effector functions inhibit immune stimulating molecules.

Embodiment 72. The method of any one of embodiments 68-71, wherein the viral producer cells comprise nucleic acid encoding molecules that stimulate immune inhibitors and molecules that inhibit immune stimulating molecules.

Embodiment 73. The method of any one of embodiments 68-72, wherein the one or more molecules providing immune effector functions includes one or more of CTLA4, B7-1, B7-2, PD-1, PD-L1, PD-L2, CD28, VISTA, TIM-3, GALS, TIGIT, CD155, LAG3, VISTA, BTLA or HVEM.

Embodiment 74. The method of any one of embodiments 68-73, wherein the viral producer cells comprise nucleic acid encoding CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-1 and PD-L2 and VISTA, or CTLA4 and PD-L1 and PD-L1 and VISTA.

Embodiment 75. The method of any one of embodiments 68-74, wherein the one or more molecules that provides immune effector functions comprises a transmembrane domain.

Embodiment 76. The method of any one of embodiments 68-75, wherein nucleic acid encoding the one or more molecules providing immune effector functions are transiently introduced to the viral producer cells.

Embodiment 77. The method of any one of embodiments 68-76, wherein nucleic acid encoding the one or more molecules providing immune effector functions is stably maintained in the viral producer cells.

Embodiment 78. The method of embodiment 77, wherein nucleic acid encoding the one or more molecules providing immune effector functions is integrated into the genome of the viral producer cell.

Embodiment 79. The method of any one of embodiments 68-78, wherein the viral producer cells comprise nucleic acid encoding one or more targeting molecules that target the vector to one or more cell types.

Embodiment 80. The method of embodiment 79, wherein the targeting molecules confer tissue specificity to the enveloped vector.

Embodiment 81. The method of embodiment 80, wherein the targeting molecule is an antibody.

Embodiment 82. The method of embodiment 71, wherein the antibody is antibody 8D7.

Embodiment 83. The method of any one of embodiments 79-82, wherein the one or more targeting molecules comprises a transmembrane domain.

Embodiment 84. The method of any one of embodiments 79-83, wherein nucleic acid encoding the one or more targeting molecules is transiently introduced to the viral producer cells.

Embodiment 85. The method of any one of embodiments 79-84, wherein nucleic acid encoding the one or more targeting molecules is stably maintained in the viral producer cells.

Embodiment 86. The method of embodiment 85, wherein nucleic acid encoding the one or more molecules targeting molecules is integrated into the genome of the viral producer cell.

Embodiment 87. The method of any one of embodiments 68-86, wherein the enveloped viral vector is an enveloped AAV vector.

Embodiment 88. The method of embodiment 87, wherein the viral producer cells comprise a) nucleic acid encoding AAV rep and cap genes, b) nucleic acid encoding an AAV viral genome comprising a transgene and at least one ITR, and c) AAV helper functions.

Embodiment 89. The method of embodiment 88, wherein the nucleic acid encoding AAV rep and cap genes and/or the AAV viral genome are transiently introduced in the producer cell line.

Embodiment 90. The method of embodiment 88, wherein the nucleic acid encoding AAV rep and cap genes and/or the AAV viral genome are stably maintained in the producer cell line.

Embodiment 91. The method of embodiment 90, wherein the nucleic acid encoding AAV rep and cap genes and/or the AAV viral genome are stably integrated into the genome of the producer cell line.

Embodiment 92. The method of any one of embodiments 88-91, wherein the rAAV genome comprises two AAV ITRs.

Embodiment 93. The method of any one of embodiments 88-92,wherein one or more AAV helper functions are provided by one or more of a plasmid, an adenovirus, a nucleic acid stably integrated into the cell genome or a herpes simples virus (HSV).

Embodiment 94. The method of any one of embodiments 88-93, wherein AAV helper functions comprise one or more of adenovirus E1A function, adenovirus E1B function, adenovirus E2A function, adenovirus E4 function and adenovirus VA function.

Embodiment 95. The method of any one of embodiments 88-93, wherein AAV helper functions comprise one or more of HSV UL5 function, HSV UL8 function, HSV UL52 function, and HSV UL29 function.

Embodiment 96. The method of any one of embodiments 68-86, wherein the enveloped viral vector is a lentiviral vector.

Embodiment 97. The method of embodiment 96, wherein the lentiviral vector is a human immunodeficiency virus, a simian immunodeficiency virus or a feline immunodeficiency virus.

Embodiment 98. The method of embodiment 96 or 97, wherein the viral producer cells comprise a) nucleic acid encoding lentiviral gag gene, b) nucleic acid encoding lentiviral pol gene, c) nucleic acid encoding a lentiviral transfer vector comprising a transgene, a 5′ long terminal repeat (LTR) and a 3′ LTR, wherein all or part of a U3 region of the 3′ LTR is replaced by a heterologous regulatory element.

Embodiment 99. The method of any one of embodiments 68-98, wherein the enveloped vector is further purified.

Embodiment 100. A kit comprising the composition of any one of embodiments 1-33.

Embodiment 101. The kit of embodiment 100 further comprising instructions for use.

Embodiment 102. A composition for use in delivering a nucleic acid to an individual in need thereof according to embodiments 34-67.

Embodiment 103. A composition for use in treating a disease or disorder to an individual in need thereof according to embodiments 34-67.

Embodiment 104. Use of the composition of any one of embodiments 1-33 in the manufacture of a medicament for delivering a nucleic acid to an individual in need thereof.

Embodiment 105. Use of the composition of any one of embodiments 1-33 in the manufacture of a medicament for treating an individual with a disease or disorder.

Embodiment 106. The use of embodiment 105, wherein the disease or disorder is myotobularin myopathy, spinal muscular atrophy, Leber's congenital amaurosis, hemophilia A, hemophilia B, choroideremia, Huntington's disease, Batten disease, Leber hereditary optic neuropathy, ornithine transcarbamylase (OTC) deficiency, Pompe disease, Fabry disease, citrullinemia type 1, phenylketonuria (PKU), adrenoleukodystrophy, sickle cell disease, or beta thalessemia.

Embodiment 107. An article of manufacture comprising the composition of any one of embodiments 1-33.

Embodiment 108. An enveloped viral vector comprising a viral particle surrounded by an envelope, wherein the viral particle comprises a heterologous transgene, and the envelope comprises a lipid bilayer and one or more immunosuppressive molecules.

Embodiment 109. The enveloped viral vector of embodiment 108, wherein the enveloped virus has reduced immunogenicity compared to a vector of the same type without immunosuppressive molecules in the lipid bilayer.

Embodiment 110. The enveloped viral vector of embodiment 108 or 109, wherein the one or more immunosuppressive molecules comprise one or more immune checkpoint proteins.

Embodiment 111. The enveloped viral vector of any one of embodiments 108-110, wherein the one or more immunosuppressive molecules comprise one or more of CTLA4, B7-1, B7-2, PD-1, PD-L1, PD-L2, CD28, VISTA, TIM-3, GALS, TIGIT, CD155, LAG3, VISTA, BTLA or HVEM.

Embodiment 112. The enveloped viral vector of any one of embodiments 108-111, wherein the envelope comprises two or more, three or more, or four or more different immunosuppressive molecules; or comprises two or more, three or more, or four or more different checkpoint proteins.

Embodiment 113. The enveloped viral vector of any one of embodiments 108-112, wherein the envelope comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

Embodiment 114. The enveloped viral vector of any one of embodiments 108-113, wherein one or more of the immunosuppressive molecules comprises a transmembrane domain.

Embodiment 115. The enveloped viral vector of any one of embodiments 108-114, wherein the envelope further comprises a targeting molecule.

Embodiment 116. The enveloped viral vector of embodiment 115, wherein the targeting molecule confers cell- or tissue-specificity to the enveloped vector.

Embodiment 117. The enveloped viral vector of embodiment 116, wherein the targeting molecule is an antibody.

Embodiment 118. The enveloped viral vector of any one of embodiments 115-117, wherein the one or more targeting molecules comprises a transmembrane domain.

Embodiment 119. The enveloped viral vector of any one of embodiments 108-118, wherein the envelope comprises a portion of a cell membrane from a cell comprising one or more exogenous nucleic acids encoding the one or more immunosuppressive molecules.

Embodiment 120. The enveloped viral vector of embodiment 119, wherein the viral particle comprises a viral capsid and a viral genome, and the viral genome comprises the heterologous transgene.

Embodiment 121. The enveloped viral vector of embodiment 120, wherein the heterologous transgene encodes a polypeptide.

Embodiment 122. The enveloped viral vector of embodiment 121, wherein the heterologous transgene encodes a therapeutic polypeptide or a reporter polypeptide.

Embodiment 123. The enveloped viral vector of embodiment 122, wherein the heterologous transgene encodes Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD).

Embodiment 124. The enveloped viral vector of embodiment 120, wherein the heterologous transgene encodes a therapeutic nucleic acid.

Embodiment 125. The enveloped viral vector of embodiment 124, wherein the therapeutic nucleic acid is a siRNA, miRNA, shRNA, antisense RNA, RNAzyme, or DNAzyme.

Embodiment 126. The enveloped viral vector of embodiment 120, wherein the heterologous transgene encodes one or more gene editing products.

Embodiment 127. The enveloped viral vector of embodiment 126, wherein the one or more gene editing products is an RNA-guided nuclease, a guide nucleic acid, and/or a donor nucleic acid.

Embodiment 128. The enveloped viral vector of any one of embodiments 108-127, wherein the viral particle comprises an adeno- associated viral vector (AAV).

Embodiment 129. The enveloped viral vector of embodiment 128, wherein the AAV vector comprises a capsid from human AAV serotype AAV1, AAV2, AAV3, AAV4, AAV5, AAV6, AAV7, AAV8, AAV9, AAV10, AAV11 or AAV12.

Embodiment 130. The enveloped viral vector of embodiment 128 or 129, wherein the AAV comprises an AAV viral genome comprising inverted terminal repeat (ITR) sequences wherein the AAV capsid and the AAV ITR are from the same AAV serotype or from different AAV serotypes.

Embodiment 131. The enveloped viral vector of any one of embodiments 108 or 128-130, wherein the enveloped viral vector is an enveloped AAV comprising a heterologous transgene encoding human Factor IX, and the envelope is an exosome engineered to contain CTLA-4 and PD-L1.

Embodiment 132. The enveloped viral vector of any one of embodiments 108 or 128-131, wherein the envelope is an exosome from a producer cell engineered to overexpress CTLA-4 and PD-L1.

Embodiment 133. The enveloped viral vector of any one of embodiments 108 or 128-130, wherein the enveloped viral vector is an enveloped AAV comprising a heterologous transgene encoding human Factor VIII, and the envelope is an exosome engineered to contain CTLA-4 and PD-L1.

Embodiment 134. The enveloped viral vector of embodiment 133, wherein the envelope is an exosome from a producer cell engineered to overexpress CTLA-4 and PD-L1.

Embodiment 135. The enveloped viral vector of any one of embodiments 108-127, wherein the viral particle comprises a lentiviral vector.

Embodiment 136. The enveloped viral vector of embodiment 135, wherein the lentiviral vector is a human immunodeficiency virus, a simian immunodeficiency virus or a feline immunodeficiency virus.

Embodiment 137. The enveloped viral vector of any of embodiments 108-136, wherein the vector when administered as a single dose to a subject provides transgene expression levels 3-weeks following administration to a subject that are increased by about 50% or more as compared to transgene expression produced by administration of a non-enveloped viral vector of the same type in the same amount and under the same conditions.

Embodiment 138. The enveloped viral vector of any of embodiments 108-137, wherein the vector provides transgene expression levels 3-weeks following administration as a single dose to a subject that are increased by about 20% or more as compared to the transgene expression produced by administration of an enveloped viral vector of the same type in the same amount without the immunosuppressive molecules under the same conditions.

Embodiment 139. A composition comprising the enveloped viral vector of any one of embodiments 108-138 and one or more pharmaceutically acceptable excipients.

Embodiment 140. A method of delivering a transgene to a cell or subject, the method comprising administering to the cell or subject an enveloped viral vector of any one of embodiments 108-138, or a composition of embodiment 139.

Embodiment 141. The method of embodiment 140, wherein the subject has a disease or condition that can be treated by delivery and expression of the transgene.

Embodiment 142. A method of treating a disease or disorder in a subject, the method comprising administering to the subject an enveloped viral vector of any one of embodiments 108-138, or a composition of embodiment 139.

Embodiment 143. The method of any one embodiments 140-142, wherein the subject is a human.

Embodiment 144. The method of any one of embodiments 141-143, wherein the disease or disorder is monogenic disease.

Embodiment 145. The method of any one of embodiments 141-143, wherein the disease or disorder is myotobularin myopathy, spinal muscular atrophy, Leber's congenital amaurosis, hemophilia A, hemophilia B, choroideremia, Huntington's disease, Batten disease, Leber hereditary optic neuropathy, ornithine transcarbamylase (OTC) deficiency, Pompe disease, Fabry disease, citrullinemia type 1, phenylketonuria (PKU), adrenoleukodystrophy, sickle cell disease, Niemann-Pick disease, or beta thalessemia.

Embodiment 146. The method of any one of embodiments 141-143, wherein the disease or disorder is hemophilia A or hemophilia B.

Embodiment 147. The method of any one of embodiments 141-143, wherein the subject has hemophilia B, the enveloped viral vector comprises an AAV comprising a heterologous transgene encoding Factor IX, and the envelope is an exosome engineered to contain CTLA-4 and PD-L1.

Embodiment 148. The method of any one of embodiments 141-143, wherein the subject has hemophilia A, the enveloped viral vector comprises an enveloped AAV comprising a heterologous transgene encoding human Factor VIII, and the envelope is an exosome engineered to contain CTLA-4 and PD-L1.

Embodiment 149. The method of embodiment 147 or 148, wherein the envelope is an exosome from a producer cell engineered to overexpress CTLA-4 and PD-L1.

Embodiment 150. The method of any of embodiments 140-149, wherein the method comprises administering two or more doses of the enveloped viral vector to the subject with an interval of 1 day or more between each dose.

Embodiment 151. A method of producing an enveloped viral vector of any of embodiments 108-138, the method comprising culturing viral producer cells in vitro under conditions to generate enveloped viral particles, wherein the viral producer cells comprise nucleic acids encoding one or more one or more membrane-bound immunosuppressive molecules, and collecting the enveloped viral vectors.

Embodiment 152. The method of embodiment 151, wherein the viral producer cells comprise exogenous nucleic acids encoding the membrane-bound immunosuppressive molecules.

Embodiment 153. The method of embodiment 151 or 152, wherein the viral producer cells comprise heterologous nucleic acids encoding the membrane-bound immunosuppressive molecules.

Embodiment 154. The method of any one of embodiments 151-153, wherein the membrane-bound immunosuppressive molecules comprise one or more of CTLA4, B7-1, B7-2, PD-1, PD-L1, PD-L2, CD28, VISTA, TIM-3, GALS, TIGIT, CD155, LAGS, VISTA, BTLA or HVEM.

Embodiment 155. The method of any one of embodiments 151-153, wherein the membrane-bound immunosuppressive molecules comprise CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-L1 and VISTA.

Embodiment 156. The method of any one of embodiments 151-155, wherein the viral producer cells comprise heterologous nucleic acids encoding CTLA-4 and PD-L1.

Embodiment 157. The method of any one of embodiments 151-156, wherein the nucleic acids encoding one or more one or more membrane-bound immunosuppressive molecules are transiently introduced to the viral producer cells.

Embodiment 158. The method of any one of embodiments 151-156, wherein the nucleic acids encoding one or more one or more membrane-bound immunosuppressive molecules are stably maintained in the viral producer cells.

Embodiment 159. The method of embodiment 158, wherein the nucleic acids encoding one or more one or more membrane-bound immunosuppressive molecules are integrated into the genome of the viral producer cell.

Embodiment 160. The method of any one of embodiments 151-159, wherein the viral producer cells comprise nucleic acids encoding one or more targeting molecules.

Embodiment 161. The method of any one of embodiments 151-160, wherein the enveloped viral vector is an enveloped AAV vector.

Embodiment 162. The method of embodiment 161, wherein the viral producer cells comprise nucleic acid encoding AAV rep and cap genes, nucleic acid encoding an AAV viral genome comprising a transgene and at least one ITR, and AAV helper functions.

Embodiment 163. The method of embodiment 162, wherein the nucleic acid encoding AAV rep and cap genes and/or the AAV viral genome are transiently introduced in the producer cell line.

Embodiment 164. The method of embodiment 162, wherein the nucleic acid encoding AAV rep and cap genes and/or the AAV viral genome are stably maintained in the producer cell line.

Embodiment 165. The method of embodiment 164, wherein the nucleic acid encoding AAV rep and cap genes and/or the AAV viral genome are stably integrated into the genome of the producer cell line.

Embodiment 166. The method of any one of embodiments 151-165, wherein one or more AAV helper functions are provided by one or more of a plasmid, an adenovirus, a nucleic acid stably integrated into the cell genome or a herpes simples virus (HSV).

Embodiment 167. The method of any one of embodiments 151-166, wherein AAV helper functions comprise one or more of adenovirus E1A function, adenovirus E1B function, adenovirus E2A function, adenovirus E4 function and adenovirus VA function.

Embodiment 168. The method of any one of embodiments 151-166, wherein AAV helper functions comprise one or more of HSV UL5 function, HSV UL8 function, HSV UL52 function, and HSV UL29 function.

Embodiment 169. The method of any one of embodiments 151-160, wherein the enveloped viral vector is a lentiviral vector.

Embodiment 170. The method of embodiment 169, wherein the lentiviral vector is a human immunodeficiency virus, a simian immunodeficiency virus or a feline immunodeficiency virus.

Embodiment 171. The method of embodiment 169 or 170, wherein the viral producer cells comprise nucleic acid encoding lentiviral gag gene, nucleic acid encoding lentiviral pol gene, nucleic acid encoding a lentiviral transfer vector comprising a transgene, a 5′ long terminal repeat (LTR) and a 3′ LTR, wherein all or part of a U3 region of the 3′ LTR is replaced by a heterologous regulatory element, a primer binding site, all or part of the GAG gene, a central polypurine tract, synthetic stop codons in the GAG sequence, rev responsive element, and an env splice acceptor.

Embodiment 172. The method of any one of embodiments 151-171, wherein the enveloped vector is further purified.

Embodiment 173. A kit comprising the enveloped viral vector of any one of embodiments 108-138 or composition of embodiment 139.

Embodiment 174. The kit of embodiment 173 further comprising instructions for use.

Embodiment 175. An enveloped viral vector of any of embodiments 108-138 or composition of embodiment 139 for use in delivering a nucleic acid to a subject.

Embodiment 176. An enveloped viral vector of any of embodiments 108-138 or composition of embodiment 139 for use in treating a disease or disorder in a subject.

Embodiment 177. The enveloped viral vector or composition of embodiment 175 or 176 for use in delivering a nucleic acid to a subject in accordance with any of embodiments 140-43.

Embodiment 178. Use of the enveloped viral vector of any one of embodiments 108-138 or composition of embodiment 139 in the manufacture of a medicament for delivering a nucleic acid to an individual in need thereof.

Embodiment 179. Use of the enveloped viral vector of any one of embodiments 108-138 or composition of embodiment 139 in the manufacture of a medicament for treating an individual with a disease or disorder.

Embodiment 180. The use of embodiment 179, wherein the disease or disorder is myotobularin myopathy, spinal muscular atrophy, Leber's congenital amaurosis, hemophilia A, hemophilia B, choroideremia, Huntington's disease, Batten disease, Leber hereditary optic neuropathy, ornithine transcarbamylase (OTC) deficiency, Pompe disease, Fabry disease, citrullinemia type 1, phenylketonuria (PKU), adrenoleukodystrophy, sickle cell disease, Niemann-Pick disease, or beta thalessemia.

Embodiment 181. The use of embodiment 180, wherein the disease or disorder is hemophilia A or hemophilia B.

Embodiment 182. An article of manufacture comprising the enveloped viral vector of any one of embodiments 108-138 or composition of embodiment 139.

### EXAMPLES

### Example 1: Determination of Reduction of Anti-AAV Immune Responses

A series of experiments are undertaken in cells to demonstrate the invention. A mixed lymphocyte reaction (MLR) using PBMCs purified from AAV positive individuals is to determine how much effector vectors can reduce capsid specific immune responses as compared to serotype matched non-enveloped vectors. Similarly, an MLR is used to test whether effector vectors can inhibit the T cell response to therapeutic protein, as compared to non-enveloped vectors. This second MLR is performed as follows: antigen presenting cells are first incubated with therapeutic protein, then PBMCs (containing T and B cells) are added in the presence of effector vectors or serotype matched non-enveloped vectors. T Cell activation is measured using FACS analysis to count total T cells including CD3+, CD4+, CD8+, CD25+ (IL2R), and FoxP3+. A neutralizing antibody assay is done using serum from individuals tested positive for anti AAV capsid antibodies. The assay is performed as describe in Meliani et al. (2015) Hum Gene Ther Methods, 26:45-53.

### Example 2: Vector Production

AAVs were produced using producer cells transfected with AAV production plasmids to express the vector. Enveloped AAVs are is shed into the culture media along with a portion of the cell membrane (envelope), and were collected from culture media via a method that does not remove the envelope. Non-enveloped AAV were obtained by lysing producer cells to collect non-enveloped viral particles.

In greater detail, Standard (non-enveloped) AAV (referred to as “standard” or “std” vector in the results and figures) and Enveloped AAV vectors (referred to as “exo” vector in the results and figures) were produced in HEK293T cells as described in Simonelli et al. (2010) Molecular Therapy, 18(3): 643-650. The same AAV production plasmids were for both vector types. The vector genome plasmid (pAAV.MCS.cb.Hu FIX), contained the human Factor IX gene as described Nathwani et al. (2011) N Engl J Med, 365: 2357-65. Packaging, and helper plasmids were those used previously (id.). Production plasmids were transfected into 293T cells using PEI as described Melaini et al. (2017) Blood Advances, 1(23): 2019-31, and purified as described in Nathwani et al. (2011) N Engl J Med, 365: 2357-65. These preps were generated from 24×150 mm tissue culture dishes of 293T producer cells.

Producer cell culture was centrifuged, and producer cells separated from the supernatant. Enveloped AAV was isolated and purified from the supernatant using 2-step ultracentrifugation, and resuspended in PBS, resulting in a population of Enveloped AAV particles with an average particle size of about 100 nm. Standard (non-enveloped) AAV was harvested from the producer cells by lysing the cells in a cell lysis buffer followed by purification using a standard iodixanol gradient protocol (Melaini et al. (2017) Blood Advances, 1(23): 2019-31). Additional details of the protocol and vector yield are shown in Table 1.

TABLE 1
Production of vectors

| | | | | | | Total | | total | |
|---|---|---|---|---|---|---|---|---|---|
| | Helper | Production Plasmid Quantity | | | | DNA (μg) | | volume | |
| | Plasmid | Transfected (μg) | | | | per | | purified | |
| | Amount | (mix and aliquot into 12 plates) | | | | 150 mm | VG titer | vector | total |
| Vector Type | (μg) | AAV2/8 | hFIX | mPDL1 | mCTL4 | dish | (vg/mL) | (mL) | yield |
| Enveloped | 600 | 300 | 300 | 0 | 0 | 100 | 2.39E+12 | 0.5 | 1.20E+12 |
| (Exo-AAV8- | | | | | | | | | |
| hFIX) | | | | | | | | | |
| EVADER | 400 | 100 | 100 | 200 | 200 | | 2.70E+11 | 0.5 | 1.35E+11 |
| (EV-AAV8- | | | | | | | | | |
| hFIX) | | | | | | | | | |
| Standard | 600 | 300 | 300 | 0 | 0 | | 1.67E+13 | 0.5 | 8.35E+12 |
| (AAV8-hFIX) | | | | | | | | | |

Enveloped vectors with an envelope comprising CTLA-4 and PD-L1 (referred to in the results and figures as “Evader” or “Effector” vectors or with the designation “EF”) were produced in two batches using the same method as used for the Enveloped AAV, except that the producer HEK293T cells were co-transfected with pCMV.mCTLA-4 and pCMV.mPDL-1 expression vectors in addition to the AAV production plasmids. pCMV.mCTLA-4 contains the murine CTLA-4 cDNA sequence driven by a CMV promoter (Sino Biological catalog # MG50503-UT). pCMV.mPDL-1 contains the murine PDL-1 cDNA sequence driven by a CMV promoter (Sino Biological catalog # MG50010-M). A total of 2 preps of 24×150 mm tissue culture dishes were prepared. Additional details of the protocol and vector yield is shown in Table 1.

To confirm whether purified vectors had envelopes, a western blot was performed using an anti-CD9 antibody. CD9 is used as a marker to indicate the presence of envelope derived from produced cells. Both Enveloped AAV and EVADER vectors contained CD9 at the predicted size of about 25 KDa. As expected, Standard (non-enveloped) AAV8-FIX did not contain envelope components as evidenced by the absence of CD9.

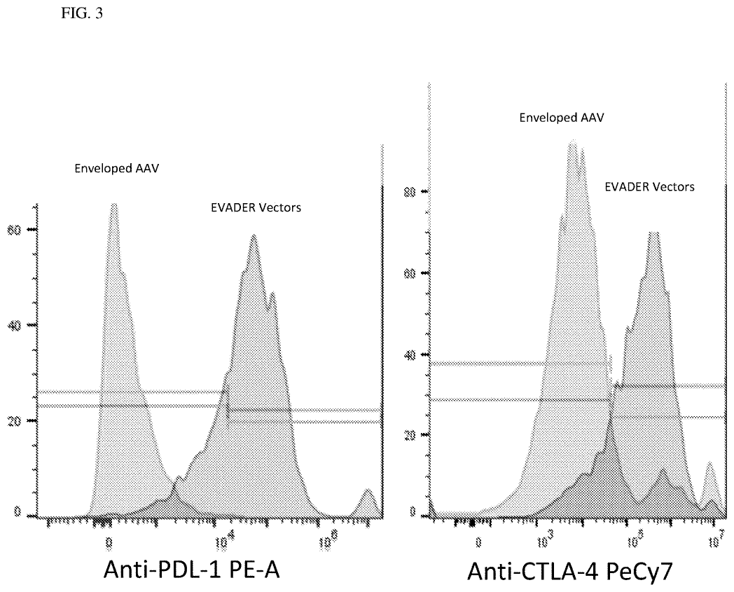

The levels of murine CTLA-4 and PDL-1 on EVADER and Enveloped AAVs were quantified using bead based FACS analysis using fluorescent-labelled antibodies: anti-murine CTLA-4 (anti-CTLA-4 PECy7, Abcam catalog number ab134090) and anti-murine PDL-1 (anti-PDL-1-PE-A, Abcam catalog number ab213480). FACS Analysis revealed that EVADER vectors had high levels of both CTLA-4 and PDL-1 (83.6% and 75.3%, respectively) on the surface as shown in FIG. 3, wherein EVADER histogram shift to the right in each figure indicates that most of the particles are positive for CTLA-4 and PD-L1, respectively, as compared to Enveloped AAV.

### Example 3: In Vivo Gene Transfer in Mice

The following example illustrates the use of the vectors produced in Example 2 for gene transfer in vivo in C57Bl/6 Mice.

C57Bl/6 Mice (seven male and seven female) were injected intravenously with 1×109 vector genomes. Dosing groups included: 1) PBS only (vehicle control), 2) AAV8-hFIX, 3) Exo-AAV8-hFIX, and 4) EV-AAV8-hFIX.

At week three post-dosing, mice were bled and analyzed for (a) human FIX levels (VisuLize™ Factor IX (FIX) Antigen Kit, Affinity Biologicals), (b) AAV8-binding antibodies (BAb) by ELISA using anti-AAV8 IgG, and (c) AAV8-neutralizing antibodies (NAb) using a neutralizing antibody assay (Meliani et al. (2015) Hum Gene Ther Methods, 26:45-53). The in-vitro neutralizing assay is used to measure the titer of antibodies that prevent from test AAV vectors infecting target cells. Briefly, the assay entails incubating an optimized multiplicity of infection (MOI) of test vector containing a reporter gene such as Luciferase, with serial dilutions of test antibodies, then allowing the vector to infect a permissive target cell. The amount of fluorescence from infected cells is measured after 24 hours and indicates the titer of neutralizing antibodies. The neutralizing titer of the sample is determined as the first dilution at which 50% or greater inhibition of the luciferase expression is measured.

Also at week three post-dosing, two male and two female mice from each group were sacrificed and livers from animals were analyzed for vector genome copy number (VGCN) per cell by qPCR. Tissue DNA was extracted from whole organ using the Magna Pure 96 DNA and viral NA small volume kit (Roche Diagnostics, Indianapolis IN) according to the manufacturer's instructions. Vector genome copy number was quantified by TaqMan real-time PCR with the ABI PRISM 7900 HT sequence detector (Thermo Fisher Scientific, Waltham, MA). The mouse titin gene was used as normalizer. The primers and probes used for the quantification were as follow:

hAAT promoter:
forward
(SEQ ID NO: 5)
5′GGCGGGCGACTCAGATC-3′,
reverse
(SEQ ID NO: 6)
5′-GGGAGGCTGCTGGTGAATATT-3′
probe FAM
(SEQ ID NO: 7)
5′-AGCCCCTGTTTGCTCCTCCGATAACTG-3′
Titin:
forward
(SEQ ID NO: 8)
5′-AAAACGAGCAGTGACGTGAGC-3′,
reverse
(SEQ ID NO: 9)
5′-TTCAGTCATGCTGCTAGCGC-3′
probe VIC
(SEQ ID NO: 10)
5′-TGCACGGAAGCGTCTCGTCTCAGTC-3′

The remaining animals were then (three weeks post-dosing) administered 1×1010 vg of the same AAV vector that was initially administered for each dose group. At week six, mice were again bled and analyzed for human FIX levels, AAV8-binding antibodies (BAb), and AAV8-neutralizing antibodies (NAb) by the same protocols. All remaining animals were then sacrificed and livers from animals were analyzed for vector genomes per cell by qPCR using the prior protocol.

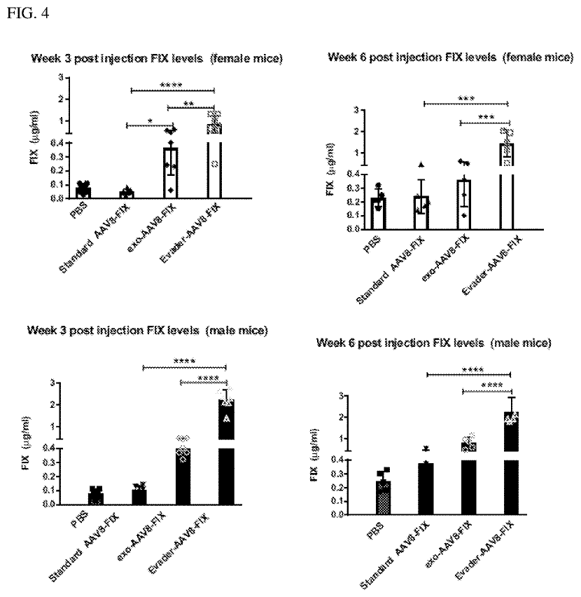

An increase in blood level of Factor IX (FIX) as compared to control animals is indicative of successful gene transfer and expression, where control animals received PBS rather than vector. As shown in FIG. 4, blood levels of FIX were significantly higher in mice treated with EV-AAV8-hFIX than in mice treated with the standard enveloped or non-enveloped virus. This was observed at both the three-week and six-week time points. The difference between Factor IX levels in male and female mice are due to a well-established animal model artifact where male mice traditionally transfect with AAV vectors at higher efficiencies in the liver than female mice. This gender based difference in transduction efficiency is an artifact of the mouse model and does not occur in humans. For the purpose of this data only male mice are considered. The variation in Factor IX levels between weeks 3 and 6 from control mice that received PBS was due to day to day variability of the assay near the limit of detection. Mice in groups that received both PBS and standard AAV showed comparable Factor IX levels at week 3 which was about 0.1 μg/mL. At week 3, the levels in EV-AAV8-hFIX treated mice were about 22 times higher than mice treated with standard non-enveloped AAV, and about 5.6 times higher than enveloped AAV without immunosuppressive molecules in the envelope. Similarly, at week 6, FIX levels in EV-AAV8-hFIX treated mice were about 20 times higher than mice treated with standard non-enveloped AAV, and about 5 times higher than enveloped AAV without immunosuppressive molecules in the envelope. These results demonstrate that the EVADER vector comprising immunosuppressive molecules in the envelope provided significantly enhanced factor IX gene expression in vivo as compared to standard AAV or standard enveloped AAV.

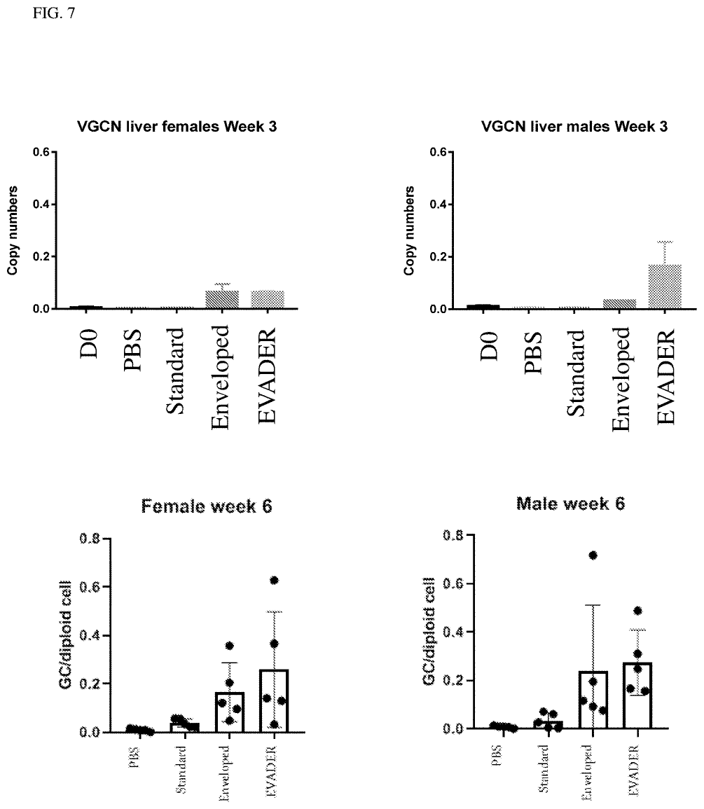

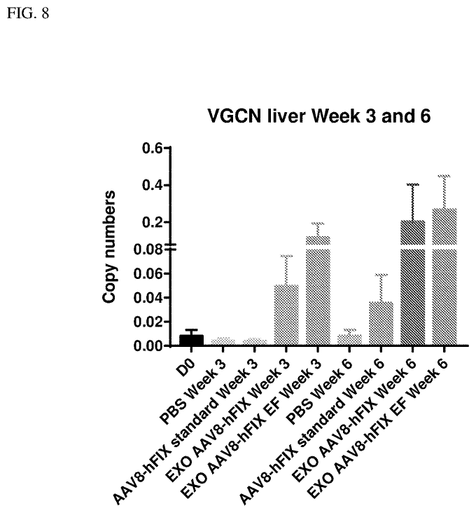

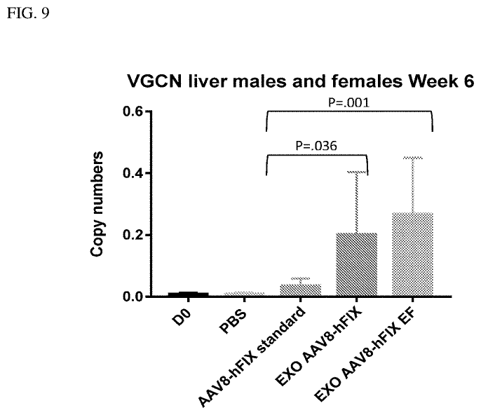

FIGS. 7-9 shows the number of viral genomes per cell in the livers of sacrificed animals. Again, the EV-AAV8-hFIX treated mice showed a higher number of viral genomes in the liver as compared to the other treatment groups at the six-week time point, indicating greater efficiency in transduction as compared to standard AAV.

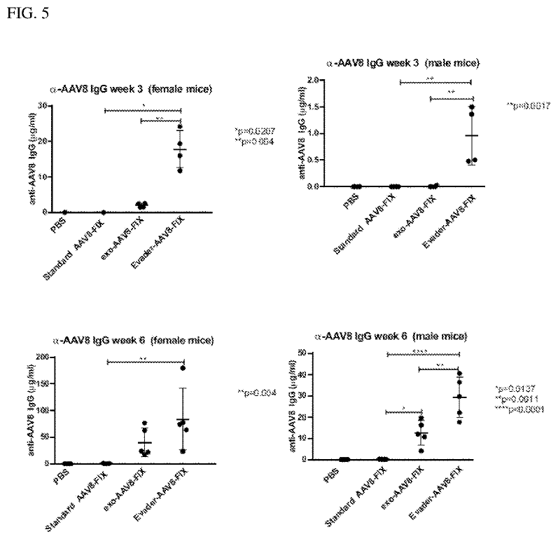

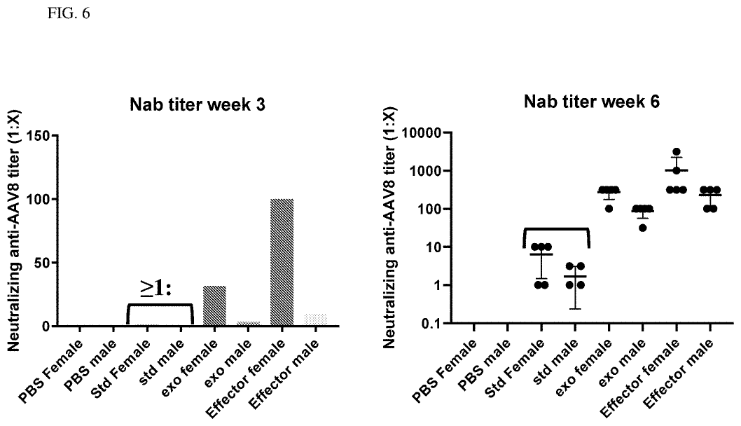

FIGS. 5 and 6 show the levels of total AAV-binding antibodies and neutralizing AAV antibodies in the blood of the treated mice. It was observed that mice treated with EV-AAV8-hFIX had higher antibody levels than mice treated with the other vectors. The vectors were analyzed for endotoxin levels (TOXINSENSOR™ Chromogenic LAL Endotoxin Assay Kit by Genscript), since endotoxin is a potent stimulator of both antibody production and inflammation, and could cause the observed increase in antibody production levels. The results are set forth in Table 2. From the results in Table 2, the amount of endotoxin administered to mice was calculated by normalizing the amount of endotoxin to the dose received by standard AAV8-FIX mice. Relative endotoxin levels administered for doses 1 and 2 were similar, so only the relative amounts for the first dose are shown in FIG. 4. It was calculated that mice treated with the EV-AAV8-hFIX vector received ˜300-fold higher endotoxin levels per dose per animal compared to the standard AAV8-hFIX vector, and mice treated with exo-AAV8-hFIX received ˜50-fold higher endotoxin levels per dose per animal as compared to mice treated with standard AAV8-FIX. Thus, it is likely that the higher antibody titers in the EV-AAV8-hFIX treated mice are due to increased endotoxin levels in this experiment.

TABLE 2

| | Standard | Enveloped | EVADER | EVADER |
|---|---|---|---|---|
| Test | AAV8-FIX | AAV8-FIX | (1st Dose) | (2nd Dose) |
| Endotoxin (EU/mL) | 0.1128 | 0.9348 | 0.5840 | 0.3983 |
| VG Titer (VG/mL) | 1.67E+13 | 2.39E+12 | 2.70E+11 | N/A |

Despite the increased BAb and NAb levels in EV-AAV8-hFIX treated mice, the EV-AAV8-hFIX vector was able to deliver the hFIX transgene and increase FIX expression significantly as compared to all other treatment groups. This suggests that the presence of immunosuppressive molecules in the envelope of the EV-AAV8-hFIX vector has a significant positive effect on transgene expression.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosed subject matter and does not pose a limitation on the scope of the disclosure unless otherwise claimed. No language in the specification should be construed as indicating any non-claimed element as essential to the practice of the subject matter disclosed herein.

Embodiments are described herein, including the best mode of operation. Variations of those embodiments may become apparent to those of ordinary skill in the art upon reading the foregoing description, and such variations are contemplated by applicant. Accordingly, disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

SEQUENCES

```
human F8 (UniProtKB-Q2VF45), SQ-FVIII variant of a B-domain-deleted (BDD)
                10 20 30 40 50
MQIELSTCFF LCLLRFCFSA TRRYYLGAVE LSWDYMQSDL GELPVDARFP
                60 70 80 90 100
PRVPKSFPFN TSVVYKKTLF VEFTDHLFNI AKPRPPWMGL LGPTIQAEVY
              110 120 130 140 150
DTVVITLKNM ASHPVSLHAV GVSYWKASEG AEYDDQTSQR EKEDDKVFPG
              160 170 180 190 200
GSHTYVWQVL KENGPMASDP LCLTYSYLSH VDLVKDLNSG LIGALLVCRE
              210 220 230 240 250
GSLAKEKTQT LHKFILLFAV FDEGKSWHSE TKNSLMQDRD AASARAWPKM
              260 270 280 290 300
HTVNGYVNRS LPGLIGCHRK SVYWHVIGMG TTPEVHSIFL EGHTFLVRNH
              310 320 330 340 350
RQASLEISPI TFLTAQTLLM DLGQFLLFCH ISSHQHDGME AYVKVDSCPE
              360 370 380 390 400
EPQLRMKNNE EAEDYDDDLT DSEMDVVRFD DDNSPSFIQI RSVAKKHPKT
              410 420 430 440 450
WVHYIAAEEE DWDYAPLVLA PDDRSYKSQY LNNGPQRIGR KYKKMRFMAY
              460 470 480 490 500
TDETFKTREA IQHESGILGP LLYGEVGDTL LIIFKNQASR PYNIYPHGIT
              510 520 520 540 550
DVRPLYSRRL PKGVKHLKDF PILPGEIFKY KWTVTVEDGP TKSDPRCLTR
              560 570 580 590 600
YYSSFVNMER DLASGLIGPL LICYKESVDQ RGNQIMSDKR NVILFSVFDE
              610 620 630 640 650
NRSWYLTENI QRFLPNPAGV QLEDPEFQAS NIMHSINGYV FDSLQLSVCL
              660 670 680 690 700
HEVAYWYILS IGAQTDFLSV FFSGYTFKHK MVYEDTLTLF PFSGETVFMS
              710 720 730 740 750
MENPGLWILG CHNSDFRNRG MTALLKVSSC DKNTGDYYED SYEDISAYLL
              760 770 780 790 800
SKNNAIEPRS FSQNSRHPST RQKQFNATTI PENDIEKTDP WFAHRTPMPK
              810 820 830 840 850
IQNVSSSDLL MLLRQSPTPH GLSLSDLQEA KYETFSDDPS PGAIDSNNSL
              860 870 880 890 900
SEMTHFRPQL HHSGDMVFTP ESGLQLRLNE KLGTTAATEL KKLDFKVSST
              910 920 930 940 950
SNNLISTIPS DNLAAGTDNT SSLGPPSMPV HYDSQLDTTL FGKKSSPLTE
              960 970 980 990 1000
SGGPLSLSEE NNDSKLLESG LMNSQESSWG KNVSSTESGR LFKGKRAHGP
            1010 1020 1030 1040 1050
ALLTKDNALF KVSISLLKTN KTSNNSATNR KTHIDGPSLL IENSPSVWQN
            1060 1070 1080 1090 1100
ILESDTEFKK VTPLIHDRML MDKNATALRL NHMSNKTTSS KNMEMVQQKK
            1110 1120 1130 1140 1150
EGPIPPDAQN PDMSFFKMLF LPESARWIQR THGKNSLNSG QGPSPKQLVS
            1160 1170 1180 1190 1200
LGPEKSVEGQ NFLSEKNKVV VGKGEFTKDV GLKEMVFPSS RNLFLTNLDN
            1210 1220 1230 1240 1250
LHENNTHNQE KKIQEEIEKK ETLIQENVVL PQIHTVTGTK NFMKNLFLLS
            1260 1270 1280 1290 1300
TRQNVEGSYD GAYAPVLQDF RSLNDSTNRT KKHTAHFSKK GEEENLEGLG
            1310 1320 1330 1340 1350
NQTKQIVEKY ACTTRISPNT SQQNFVTQRS KRALKQFRLP LEETELEKRI
            1360 1370 1380 1390 1400
IVDDTSTQWS KNMKHLTPST LTQIDYNEKE KGAITQSPLS DCLTRSHSIP
            1410 1420 1430 1440 1450
QANRSPLPIA KVSSFPSIRP IYLTRVLFQD NSSHLPAASY RKKDSGVQES
            1460 1470 1480 1490 1500
SHFLQGAKKN NLSLAILTLE MTGDQREVGS LGTSATNSVT YKKVENTVLP
            1510 1520 1530 1540 1550
KPDLPKTSGK VELLPKVHIY QKDLFPTETS NGSPGHLDLV EGSLLQGTEG
            1560 1570 1580 1590 1600
AIKWNEANRP GKVPFLRVAT ESSAKTPSKL LDPLAWDNHY GTQIPKEEWK
            1610 1620 1630 1640 1650
SQEKSPEKTA FKKKDTILSL NACESNHAIA AINEGQNKPE IEVTWAKQGR
            1660 1670 1680 1690 1700
TERLCSQNPP VLKRHQREIT RTTLQSDQEE IDYDDTISVE MKKEDFDIYD
            1710 1720 1730 1740 1750
EDENQSPRSF QKKTRHYFIA AVERLWDYGM SSSPHVLRNR AQSGSVPQFK
            1760 1770 1780 1790 1800
KVVFQEFTDG SFTQPLYRGE LNEHLGLLGP YIRAEVEDNI MVTFRNQASR
            1810 1820 1830 1840 1850
PYSFYSSLIS YEEDQRQGAE PRKNFVKPNE TKTYFWKVQH HMAPTKDEFD
            1860 1870 1880 1890 1900
CKAWAYFSDV DLEKDVHSGL IGPLLVCHTN TLNPAHGRQV TVQEFALFFT
            1910 1920 1930 1940 1950
IFDETKSWYF TENMERNCRA PCNIQMEDPT FKENYRFHAI NGYIMDTLPG
            1960 1970 1980 1990 2000
LVMAQDQRIR WYLLSMGSNE NIHSIHFSGH VFTVRKKEEY KMALYNLYPG
            2010 2020 2030 2040 2050
VFETVEMLPS KAGIWRVECL IGEHLHAGMS TLFLVYSNKC QTPLGMASGH
            2060 2070 2080 2090 2100
IRDFQITASG QYGQWAPKLA RLHYSGSINA WSTKEPFSWI KVDLLAPMII
            2110 2120 2130 2140 2150
HGIKTQGARQ KESSLYISQF IIMYSLDGKK WQTYRGNSTG TLMVFFGNVD
            2160 2170 2180 2190 2200
SSGIKHNIFN PPIIARYIRL HPTHYSIRST LRMELMGCDL NSCSMPLGME
            2210 2220 2230 2240 2250
SKAISDAQIT ASSYFTNMFA TWSPSKARLH LQGRSNAWRP QVNNPKEWLQ
            2260 2270 2280 2290 2300
VDFQKTMKVT GVTTQGVKSL LTSMYVKEFL ISSSQDGHQW TLFFQNGKVK
            2310 2320 2330 2340 2350
VFQGNQDSFT PVVNSLDPPL LTRYLRIHPQ SWVHQIALRM EVLGCEAQDL
Y
(SEQ ID NO: 1)
human Factor IX UniProtKB-P00740
                10 20 30 40 50
MQRVNMIMAE SPGLITICLL GYLLSAECTV FLDHENANKI LNRPKRYNSG
                60 70 80 90 100
KLEEFVQGNL ERECMEEKCS FEEAREVFEN TERTTEFWKQ YVDGDQCESN
              110 120 130 140 150
PCLNGGSCKD DINSYECWCP FGFEGKNCEL DVTCNIKNGR CEQFCKNSAD
              160 170 180 190 200
NKVVCSCTEG YRLAENQKSC EPAVPFPCGR VSVSQTSKLT RAETVFPDVD
              210 220 230 240 250
YVNSTEAETI LDNITQSTQS FNDFIRVVGG EDAKPGQFPW QVVLNGKVDA
              260 270 280 290 300
FCGGSIVNEK WIVTAAHCVE TGVKITVVAG EHNIEETEHT EQKRNVIRII
              310 320 330 340 350
PHHNYNAAIN KYNHDIALLE LDEPLVLNSY VTPICIADKE YTNIFLKFGS
              360 370 380 390 400
GYVSGWGRVF HKGRSALVLQ YLRVPLVDRA TCLRSTKFTI YNNMFCAGFH
              410 420 430 440 450
EGGRDSCQGD SGGPHVTEVE GTSFLTGIIS WGEECAMKGK YGIYTKVSRY
              460
VNWIKEKTKL T
(SEQ ID NO: 2)
Human CTLA-4: NCBI Reference Sequence: NP_005205.2
                10 20 30 40 50
MACLGFQRHK AQLNLATRTW PCTLLFFLLF IPVFCKAMHV AQPAVVLASS
                60 70 80 90 100
RGIASFVCEY ASPGKATEVR VTVLRQADSQ VTEVCAATYM MGNELTFLDD
              110 120 130 140 150
SICTGTSSGN QVNLTIQGLR AMDTGLYICK VELMYPPPYY LGIGNGTQIY
              160 170 180 190 200
VIDPEPCPDS DFLLWILAAV SSGLFFYSFL LTAVSLSKML KKRSPLTTGV
              210 220
YVKMPPTEPE CEKQFQPYFI PIN
(SEQ ID NO: 3)
Human PDL-1: NCBI Reference Sequence: NP_054862.1
    1 MRIFAVFIFM TYWHLLNAFT VTVPKDLYVV EYGSNMTIEC KFPVEKQLDL AALIVYWEME
  61 DKNIIQFVHG EEDLKVQHSS YRQRARLLKD QLSLGNAALQ ITDVKLQDAG VYRCMISYGG
121 ADYKRITVKV NAPYNKINQR ILVVDPVTSE HELTCQAEGY PKAEVIWTSS DHQVLSGKTT
181 TTNSKREEKL FNVTSTLRIN TTTNEIFYCT FRRLDPEENH TAELVIPELP LAHPPNERTH
241 LVILGAILLC LGVALTFIFR LRKGRMMDVK KCGIQDTNSK KQSDTHLEET
(SEQ ID NO: 4)
```

What is claimed is:

1. An enveloped viral vector comprising a viral particle surrounded by an envelope, wherein the viral particle comprises a heterologous transgene, and the envelope comprises a lipid bilayer and one or more immunosuppressive molecules.

2. The enveloped viral vector of claim 1, wherein the enveloped virus has reduced immunogenicity compared to a vector of the same type without immunosuppressive molecules in the lipid bilayer.

3. The enveloped viral vector of claim 1 or 2, wherein the one or more immunosuppressive molecules comprise one or more immune checkpoint proteins.

4. The enveloped viral vector of any one of claims 1-3, wherein the one or more immunosuppressive molecules comprise one or more of CTLA4, B7-1, B7-2,PD-1, PD-L1, PD-L2, CD28, VISTA, TIM-3, GALS, TIGIT, CD155, LAG3, VISTA, BTLA or HVEM.

5. The enveloped viral vector of any one of claims 1-4, wherein the envelope comprises two or more, three or more, or four or more different immunosuppressive molecules; or comprises two or more, three or more, or four or more different checkpoint proteins.

6. The enveloped viral vector of any one of claims 1-5, wherein the envelope comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

7. The enveloped viral vector of any one of claims 1-6, wherein one or more of the immunosuppressive molecules comprises a transmembrane domain.

8. The enveloped viral vector of any one of claims 1-7, wherein the envelope further comprises a targeting molecule.

9. The enveloped viral vector of claim 8, wherein the targeting molecule confers cell- or tissue-specificity to the enveloped vector.

10. The enveloped viral vector of claim 9, wherein the targeting molecule is an antibody.

11. The enveloped viral vector of any one of claims 8-10, wherein the one or more targeting molecules comprises a transmembrane domain.

12. The enveloped viral vector of any one of claims 1-11, wherein the envelope comprises a portion of a cell membrane from a cell comprising one or more exogenous nucleic acids encoding the one or more immunosuppressive molecules.

13. The enveloped viral vector of claim 12, wherein the viral particle comprises a viral capsid and a viral genome, and the viral genome comprises the heterologous transgene.

14. The enveloped viral vector of claim 13, wherein the heterologous transgene encodes a polypeptide.

15. The enveloped viral vector of claim 14, wherein the heterologous transgene encodes a therapeutic polypeptide or a reporter polypeptide.

16. The enveloped viral vector of claim 13, wherein the heterologous transgene encodes Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD).

17. The enveloped viral vector of claim 13, wherein the heterologous transgene encodes a therapeutic nucleic acid.

18. The enveloped viral vector of claim 17, wherein the therapeutic nucleic acid is a siRNA, miRNA, shRNA, antisense RNA, RNAzyme, or DNAzyme.

19. The enveloped viral vector of claim 13, wherein the heterologous transgene encodes one or more gene editing products.

20. The enveloped viral vector of claim 19, wherein the one or more gene editing products is an RNA-guided nuclease, a guide nucleic acid, and/or a donor nucleic acid.

21. The enveloped viral vector of any one of claims 1-20, wherein the viral particle comprises an adeno- associated viral vector (AAV).

22. The enveloped viral vector of claim 21, wherein the AAV vector comprises a capsid from human AAV serotype AAV1, AAV2, AAV3, AAV4, AAV5, AAV6, AAV7, AAV8,AAV9, AAV10, AAV11 or AAV12.

23. The enveloped viral vector of claim 21 or 22, wherein the AAV comprises an AAV viral genome comprising inverted terminal repeat (ITR) sequences wherein the AAV capsid and the AAV ITR are from the same AAV serotype or from different AAV serotypes.

24. The enveloped viral vector of any one of claim 1 or 21-23, wherein the enveloped viral vector is an enveloped AAV comprising a heterologous transgene encoding human Factor IX, and the envelope is an exosome engineered to contain CTLA-4 and PD-L1.

25. The enveloped viral vector of any one of claim 1 or 21-24, wherein the envelope is an exosome from a producer cell engineered to overexpress CTLA-4 and PD-L1.

26. The enveloped viral vector of any one of claim 1 or 21-23, wherein the enveloped viral vector is an enveloped AAV comprising a heterologous transgene encoding human Factor VIII, and the envelope is an exosome engineered to contain CTLA-4 and PD-L1.

27. The enveloped viral vector of claim 26, wherein the envelope is an exosome from a producer cell engineered to overexpress CTLA-4 and PD-L1.

28. The enveloped viral vector of any one of claims 1-20, wherein the viral particle comprises a lentiviral vector.

29. The enveloped viral vector of claim 28, wherein the lentiviral vector is a human immunodeficiency virus, a simian immunodeficiency virus or a feline immunodeficiency virus.

30. The enveloped viral vector of any of claims 1-29, wherein the vector when administered as a single dose to a subject provides transgene expression levels 3-weeks following administration to a subject that are increased by about 50% or more as compared to transgene expression produced by administration of a non-enveloped viral vector of the same type in the same amount and under the same conditions.

31. The enveloped viral vector of any of claims 1-30, wherein the vector provides transgene expression levels 3-weeks following administration as a single dose to a subject that are increased by about 20% or more as compared to the transgene expression produced by administration of an enveloped viral vector of the same type in the same amount without the immunosuppressive molecules under the same conditions.

32. A composition comprising the enveloped viral vector of any one of claims 1-31 and one or more pharmaceutically acceptable excipients.

33. A method of delivering a transgene to a cell or subject, the method comprising administering to the cell or subject an enveloped viral vector of any one of claims 1-31, or a composition of claim 32.

34. The method of claim 33, wherein the subject has a disease or condition that can be treated by delivery and expression of the transgene.

35. A method of treating a disease or disorder in a subject, the method comprising administering to the subject an enveloped viral vector of any one of claims 1-31, or a composition of claim 32.

36. The method of any one claims 33-35, wherein the subject is a human.

37. The method of any one of claims 34-36, wherein the disease or disorder is monogenic disease.

38. The method of any one of claims 34-36, wherein the disease or disorder is myotobularin myopathy, spinal muscular atrophy, Leber's congenital amaurosis, hemophilia A, hemophilia B, choroideremia, Huntington's disease, Batten disease, Leber hereditary optic neuropathy, ornithine transcarbamylase (OTC) deficiency, Pompe disease, Fabry disease, citrullinemia type 1, phenylketonuria (PKU), adrenoleukodystrophy, sickle cell disease, Niemann-Pick disease, or beta thalessemia.

39. The method of any one of claims 34-36, wherein the disease or disorder is hemophilia A or hemophilia B.

40. The method of any one of claims 34-36, wherein the subject has hemophilia B, the enveloped viral vector comprises an AAV comprising a heterologous transgene encoding Factor IX, and the envelope is an exosome engineered to contain CTLA-4 and PD-L1.

41. The method of any one of claims 34-36, wherein the subject has hemophilia A, the enveloped viral vector comprises an enveloped AAV comprising a heterologous transgene encoding human Factor VIII, and the envelope is an exosome engineered to contain CTLA-4 and PD-L1.

42. The method of claim 40 or 41, wherein the envelope is an exosome from a producer cell engineered to overexpress CTLA-4 and PD-L1.

43. The method of any of claims 33-42, wherein the method comprises administering two or more doses of the enveloped viral vector to the subject with an interval of 1 day or more between each dose.

44. A method of producing an enveloped viral vector of any of claims 1-31, the method comprising
a) culturing viral producer cells in vitro under conditions to generate enveloped viral particles, wherein the viral producer cells comprise nucleic acids encoding one or more one or more membrane-bound immunosuppressive molecules, and
b) collecting the enveloped viral vectors.

45. The method of claim 44, wherein the viral producer cells comprise exogenous nucleic acids encoding the membrane-bound immunosuppressive molecules.

46. The method of claim 44 or 45, wherein the viral producer cells comprise heterologous nucleic acids encoding the membrane-bound immunosuppressive molecules.

47. The method of any one of claims 44-46, wherein the membrane-bound immunosuppressive molecules comprise one or more of CTLA4, B7-1, B7-2,PD-1, PD-L1, PD-L2, CD28, VISTA, TIM-3, GALS, TIGIT, CD155, LAG3, VISTA, BTLA or HVEM.

48. The method of any one of claims 44-46, wherein the membrane-bound immunosuppressive molecules comprise CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2,CTLA4 and PD-L1 and VISTA, CTLA4 and PD-L2 and VISTA, PD-L1 and PD-L2 and VISTA, or CTLA4 and PD-L1 and PD-L1 and VISTA.

49. The method of any one of claims 44-48, wherein the viral producer cells comprise heterologous nucleic acids encoding CTLA-4 and PD-L1.

50. The method of any one of claims 44-49, wherein the nucleic acids encoding one or more one or more membrane-bound immunosuppressive molecules are transiently introduced to the viral producer cells.

51. The method of any one of claims 44-49, wherein the nucleic acids encoding one or more one or more membrane-bound immunosuppressive molecules are stably maintained in the viral producer cells.

52. The method of claim 51, wherein the nucleic acids encoding one or more one or more membrane-bound immunosuppressive molecules are integrated into the genome of the viral producer cell.

53. The method of any one of claims 44-52, wherein the viral producer cells comprise nucleic acids encoding one or more targeting molecules.

54. The method of any one of claims 44-53, wherein the enveloped viral vector is an enveloped AAV vector.

55. The method of claim 54, wherein the viral producer cells comprise
c) nucleic acid encoding AAV rep and cap genes,
d) nucleic acid encoding an AAV viral genome comprising a transgene and at least one ITR, and
e) AAV helper functions.

56. The method of claim 55, wherein the nucleic acid encoding AAV rep and cap genes and/or the AAV viral genome are transiently introduced in the producer cell line.

57. The method of claim 55, wherein the nucleic acid encoding AAV rep and cap genes and/or the AAV viral genome are stably maintained in the producer cell line.

58. The method of claim 57, wherein the nucleic acid encoding AAV rep and cap genes and/or the AAV viral genome are stably integrated into the genome of the producer cell line.

59. The method of any one of claims 44-58, wherein one or more AAV helper functions are provided by one or more of a plasmid, an adenovirus, a nucleic acid stably integrated into the cell genome or a herpes simples virus (HSV).

60. The method of any one of claims 44-59, wherein AAV helper functions comprise one or more of adenovirus E1A function, adenovirus E1B function, adenovirus E2A function, adenovirus E4 function and adenovirus VA function.

61. The method of any one of claims 44-59, wherein AAV helper functions comprise one or more of HSV UL5 function, HSV UL8 function, HSV UL52 function, and HSV UL29 function.

62. The method of any one of claims 44-53, wherein the enveloped viral vector is a lentiviral vector.

63. The method of claim 62, wherein the lentiviral vector is a human immunodeficiency virus, a simian immunodeficiency virus or a feline immunodeficiency virus.

64. The method of claim 62 or 63, wherein the viral producer cells comprise
f) nucleic acid encoding lentiviral gag gene,
g) nucleic acid encoding lentiviral pol gene,
h) nucleic acid encoding a lentiviral transfer vector comprising a transgene, a 5′ long terminal repeat (LTR) and a 3′ LTR, wherein all or part of a U3 region of the 3′ LTR is replaced by a heterologous regulatory element, a primer binding site, all or part of the GAG gene, a central polypurine tract, synthetic stop codons in the GAG sequence, rev responsive element, and an env splice acceptor.

65. The method of any one of claims 44-64, wherein the enveloped vector is further purified.

66. A kit comprising the enveloped viral vector of any one of claims 1-31 or composition of claim 32.

67. The kit of claim 66 further comprising instructions for use.

68. An enveloped viral vector of any of claims 1-31 or composition of claim 32 for use in delivering a nucleic acid to a subject.

69. An enveloped viral vector of any of claims 1-31 or composition of claim 32 for use in treating a disease or disorder in a subject.

70. The enveloped viral vector or composition of claim 68 or 69 for use in delivering a nucleic acid to a subject in accordance with any of claims 33-43.

71. Use of the enveloped viral vector of any one of claims 1-31 or composition of claim 32 in the manufacture of a medicament for delivering a nucleic acid to an individual in need thereof.

72. Use of the enveloped viral vector of any one of claims 1-31 or composition of claim 32 in the manufacture of a medicament for treating an individual with a disease or disorder.

73. The use of claim 72, wherein the disease or disorder is myotobularin myopathy, spinal muscular atrophy, Leber's congenital amaurosis, hemophilia A, hemophilia B, choroideremia, Huntington's disease, Batten disease, Leber hereditary optic neuropathy, ornithine transcarbamylase (OTC) deficiency, Pompe disease, Fabry disease, citrullinemia type 1, phenylketonuria (PKU), adrenoleukodystrophy, sickle cell disease, Niemann-Pick disease, or beta thalessemia.

74. The use of claim 73, wherein the disease or disorder is hemophilia A or hemophilia B.

75. An article of manufacture comprising the enveloped viral vector of any one of claims 1-31 or composition of claim 32.
